(19) United States
(12) Patent Application Publication
(10) Pub. No.: US 2026/0027242 A1
(43) Pub. Date: Jan. 29, 2026

(54) PET RADIOLIGANDS FOR IMAGING GLUTAMINYL CYCLASE ACTIVITY

(71) Applicant: Vanderbilt University, Nashville, TN (US)

(72) Inventors: Wellington Pham, Nashville, TN (US); William Behof, Nashville, TN (US); Clayton Whitmore, Nashville, TN (US); Justin Haynes, Nashville, TN (US)

(21) Appl. No.: 19/076,507

(22) Filed: Mar. 11, 2025

Related U.S. Application Data
(60) Provisional application No. 63/563,595, filed on Mar. 11, 2024.

(51) Int. Cl. A61K 51/04

(52) U.S. Cl. CPC A61K 51/0453; A61K 2123/00

(57) ABSTRACT

Radiolabeled glutaminylcyclase (QC) inhibitors as imaging agents, in particular, but not exclusively, as medical imaging agents for the detection of neurological disorders; and pharmaceutical compositions, methods and kits for detecting neurological disorders, using the radiolabeled inhibitors.

### CROSS-REFERENCE TO RELATED APPLICATIONS

This U.S. Non-Provisional application claims priority to U.S. Provisional Application No. 63/563,595 filed Mar. 11, 2024.

### STATEMENT OF GOVERNMENT INTEREST

This invention was made with government support under R01 AG061138 awarded by the National Institute of Health. The government has certain rights in the invention.

### FIELD OF THE INVENTION

The present invention relates, generally, to the field of imaging agents, and more specifically the use of certain radiolabeled glutaminyl cyclase (QC) inhibitors as imaging agents, in particular but not exclusively as medical imaging agents for the detection of neurological disorders. The present disclosure also relates to pharmaceutical compositions comprising said radiolabeled inhibitors and to methods and kits for detecting neurological disorders.

### BACKGROUND

Alzheimer's disease (AD) is a progressive neurodegenerative disorder that primarily affects memory, thinking, and behavior. AD causes abnormal changes in the brain, including the aggregation of amyloid plaques and the accumulation of intracellular hyperphosphorylated Tau tangles, together, that lead to profound neuronal death. Accumulated AB peptide is the main component of senile plaques and derives from the proteolytic cleavage of a larger glycoprotein named amyloid precursor protein (APP). Cleavage products include amyloid B peptides with amino acid residues 1-40 (Aβ₄₀) and 1-42 (Aβ₄₂), which have been the center of research focus for several decades.

Amyloid-β peptides found in Alzheimer's disease (AD) are truncated, for example, so that a glutamine residue is present at the N-terminus, and undergo post-translational modification catalyzed by glutaminyl cyclase (QC) at the N-terminus. QC belongs to the family of metal-dependent aminoacyltransferases, which is responsible for the conversion of glutaminyl residues at the N-terminus of peptides into pyroglutaminyl peptides. QC mRNA and protein are upregulated in AD patients compared to normal aging individuals and correlate with the existence of larger concentrations of Abeta-pE3 compared to healthy controls.

QC is expressed in various tissues throughout the body, with its highest expression levels typically found in the central nervous system (CNS). QC inhibitors such as varoglutamstat (also known as PQ912) reduce the glutaminylation of amyloid-beta peptides. However, in a phase 2b clinical trial varoglutamstat (i.e., VIVIAD), it was shown to be safe and well-tolerated, with low discontinuation rates and no symptomatic ARIAs, but failed to improve cognition or function in patients with AD.

Positron emission tomography (PET) is a nuclear medicine imaging technology that provides moderate-resolution, sensitive images of the biodistribution of a radiotracer in vivo. PET uses a small amount of positron-emitting radioactive material (e.g., “¹⁵O, ¹³N, ¹¹C, and ¹⁸F, with relatively short half-lives of 2.037, 9.965, 20.39, and 109.8 min, respectively) to create detailed images of the brain to detect abnormalities associated with AD. ¹⁸F has the most ideal half-life for labeling of radiopharmaceuticals and has a unique and diverse chemistry for introduction into various molecules. In amyloid PET imaging, there are FDA-approved ¹⁸F-labeled tracers (such as Florbetapir, Flutemetamol, or Florbetaben) that have been used to detect amyloid plaques. While conventional amyloid PET imaging can detect the presence of amyloid plaques, it may not detect amyloid plaques in the early stages of AD, when amyloid buildup may be minimal or not yet detectable. Because radiolabeled QC inhibitors may provide an earlier and more direct indication of disease progression, this makes them potentially better markers for detecting AD in its preclinical or early stages, when interventions may be more effective. In addition, PET imaging with radiolabeled QC inhibitors can also provide important insights for both drug discovery and development and for potentially limiting side effects due to off-target binding.

### SUMMARY

Disclosed herein is a compound comprising formula (I), a hydrate, a solvate, a pharmaceutically acceptable salt, or combination thereof,

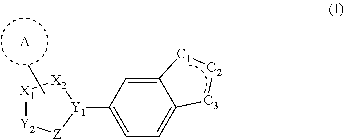

A is a 5- or 6-membered aryl ring, or a 5- or 6-membered heteroaryl ring;
a is 0 to 5;
X₁ and X₂ are each independently O, S, C(Rₓ)₂, —C(Rₓ)₂—C(Rₓ)₂—, —C(Rₓ)₂—O—, wherein at least one of X₁ and X₂ is not —C(Rₓ)₂—C(Rₓ)₂— or —C(Rₓ)₂—O—;
Y₁ is CH, CH(R_{y}), or N;
Y₂ is CH₂, CH(R_{y}), C(R_{y})₂, O, S, NH, or NR₁₀;
Z is C(R_{z})₂ or Z′(═X′)_{x′}, wherein x′ is 1 or 2;
Z′(═X′)_{x′} is C(═O), C(═CH(R_{x′})), C(═C(R_{x′})₂), C(═S), S(═O), or S(═O)₂;
B₁, B₂, and B₃ are each independently CH, C(R_{b}), or N;
C₁, C₂, and C₃ are each independently CH, C(R_{c}), or N;
each occurrence of Rₓ is independently hydrogen or a substituent;
each occurrence of Rₐ, R_{b}, R_{c}, R₁₀, R_{y}, and R_{z} is independently a substituent;
each occurrence of a substituent is:
hydroxyl, deuterated hydroxyl, thiol, deuterated thiol, cyano, halogen, isonitrile (—NC), tri C₁-C₁₂ alkylammonium, C₁-C₁₂ alkyl ketone, sulfonate, or nitro; or
a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), optionally substituted with a deuterium, hydroxyl, deuterated hydroxyl, thiol, deuterated thiol, cyano, halogen, isonitrile (—NC), tri C₁-C₁₂ alkylammonium, C₁-C₁₂ alkyl ketone, sulfonate, or nitro, or a combination thereof,
wherein any carbon-carbon single bond of the C₁-C₁₂ alkyl and the C₁-C₆ alkyl is optionally replaced by at least one carbon-carbon double or triple bond, and any methylene of the C₁-C₁₂ alkyl and the C₁-C₆ alkyl is optionally replaced by at least one O, S, NR₁₀, oxo (—C═O), imido (—C═NR₁₀), thioxo (—C═S), sulfoxo (S═O), sulfone (S(═O)₂), Se, Ge, or Si;
   wherein (i) the compound is an imaging agent and at least one substituent comprises a detectable halogen group; or (ii) the compound is a synthetic precursor of an imaging agent, wherein at least one substituent comprises a detectable halogen group.

Also disclosed is a kit comprising components A and B, wherein component A is the above-reference compound of formula (I), wherein the compound is a synthetic precursor of an imaging agent comprising a detectable halogen group, and component B is a radioactive isotope source.

Disclosed herein is a pharmaceutical composition comprising the above-reference compound of formula (I) and a pharmaceutically acceptable excipient.

Disclosed herein is a method form preparing the above-reference compound of formula (I) comprising a detectable halogen group, wherein the method comprises reacting the above-reference compound of formula (I) comprising a leaving group under conditions effective to provide the compound comprising a detectable halogen group, preferably ¹⁸F.

Disclosed herein is an imaging method for detecting amyloid β in a subject, comprising administering an effective amount of the above-reference compound of formula (I) comprising a detectable halogen group to the subject.

Disclosed herein is a method for monitoring the severity of a disease or disorder associated with the presence of amyloid in a subject, wherein the method comprises administering to the subject an effective amount of the above-reference compound of formula (I) comprising a detectable halogen group.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1 is a diagram that shows the mechanism of forming Abeta-pE3 from a truncated Abeta peptide via QC activity. Due to the loss of charged moieties, the product Abeta-pE3 is more hydrophobic and has an increased aggregation propensity. The unique implications of QC in AD represent an ideal target for imaging intervention.

FIGS. 2(A), (B), and (C) show radioisotope labeling strategy. (A) QC inhibitor PQ912; (B) Potential sites on PQ912 where a positron emitter can be labeled with either [¹¹C] or [¹⁸F] radioisotope; (C) Chemical structure of [¹⁸F]PB0822 PET radioligand as a modification from PQ912.

FIGS. 3(A) and (B) are schemes showing synthetic chemistry and characterization. (A) an optimized scheme of synthesis of the tosylate precursor for [¹⁸F]F-labeling, and the standard [¹⁹F]PB0822; (B) HPLC profiles for chiral separation of the tosylate precursor; (top): racemic compound; (middle) S-enantiomer; (bottom) R-enantiomer.

FIGS. 4(A) and (B) show examples of radioisotope labeling. (A) [¹⁸F] F labeling of the non-protected precursor using a conventional method; (B) HPLC chromatograms of the standard [¹⁹F]PB0822 co-injected with [¹⁸F]PB0822 PET radioligand, confirmed by UV and gamma detector, respectively.

FIGS. 5(A), (B), and (C) show characterization of an example of the present invention for QC binding specificity. (A) Schematic description of the assay to characterize QC inhibitory effect of PB-0822 using glutaminyl-7-amido-4-methylcoumarin as a conditioned signal readout substrate; (B) Among the tested enantiomers, only the S-[¹⁹F]PB0822 is recognized by QC and resulted in the attenuation of the fluorescent signal; (C) The assay revealed that the IC₅₀ of S-[¹⁹F]PB0822 is comparable to that of PQ912.

FIGS. 6(A) and (B) show in-vivo PET imaging of QC activity in the brains with an example of the present invention. (A) A representative axial, coronal and sagittal view of WT (n=5) and 5XFAD (n=11) brains (highlighted by discontinued white dots) after animals were injected with [18F]PB0822 radioligand 30 minutes before 20-minute PET scans. In a blocking study (bottom row), selected 5XFAD mice (n=3) were injected with standard [¹⁹F]PB0822 (compound 8) minutes prior to injecting [¹⁸F]PB0822 probe, resulting in loss of signal; (B) The detected PET signal representing specific uptake in brain regions was quantified and presented as SUV; p=0.02 between WT vs 5XFAD; p=0.002 between 5XFAD vs blocking group.

FIG. 7 shows dynamic PET imaging with a compound of the present invention. Representative PET imaging data of 5XFAD mice (n=2) to describe time-course uptake in the brain (upper panel) and whole body (lower panel) immediately after intravenous injection of [18F]PB0822 PET radioligand.

FIGS. 8(A) and (B) show immunohistochemistry to assess Abeta-pE3 levels in the brains. (A) Abeta-pE3 immunohistochemistry (red, 668 nm channel) of representative staining of Abeta-pE3 on DAPI (blue)-stained coronal brain sections (8-10 μm) using anti-Abeta-pE3 antibodies on WT versus 5XFAD mice (n=3, each, 4 slides were observed per mouse). Quantitative pixel counts obtained for WT and 5XFAD are compared; (B) Representative data of double immunostaining of Abeta and Abeta-pE3 on 5XFAD coronal brain slides (8-10 μm) and merged data. Quantitative pixel counts obtained for WT and 5XFAD are compared with significance, p<0.05.

### DETAILED DESCRIPTION

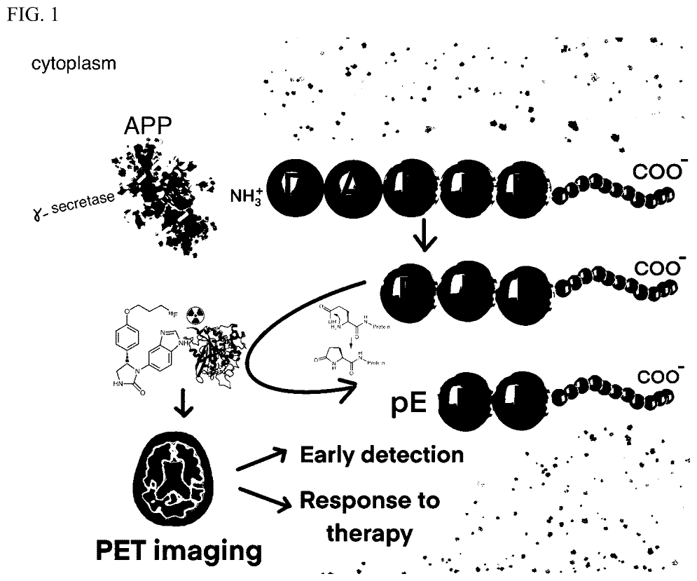

Alzheimer's disease (AD) is the most prevalent cause of dementia with unknown etiology. The cytopathologic hallmarks of AD are the extracellular amyloid-β protein (Abeta) and intracellular neurofibrillary tangles, which lead ultimately to profound neuronal toxicity and tissue atrophy. Particularly, the Abeta proteins with full-length amino acid residues 1-40 and 1-42 have been the center of research focus for several decades. However, there are other isoforms of Abeta proteins, including the N- and C-terminal truncated, as well as modified analogs. When N-terminal truncation exposes a glutamic acid residue, the amino terminus of Abeta can become cyclized into a 5-membered ring, which is a very stable moiety. This post-translational modification is catalyzed by glutaminyl cyclase (QC) to form pyroglutamate Abeta (Abeta-pE). Two Abeta-pE products of QCs found in AD brains are Abeta-pE with cyclization of the glutamate residues 3 (Abeta-pE3) and 11 (Abeta-pE11), and these have become the topic of considerable study. The QC-mediated formation of pyroglutamate leads to a more stable protein with a significant loss of electronic charges, thus enhancing the hydrophobicity of the final substrate (see FIG. 1). Without being bound by theory or mechanism, it is believed that this hydrophobicity promotes rapid conformational change and protein misfolding into the β-sheet structures, and toxic aggregation. Notably, Abeta-pE3 is found as a major species deposited in the plaques and vessels of AD and Down syndrome patients. The Abeta-pE3 is also deposited in the brains of several preclinical animal models, albeit at the later stages compared to human cases. Additionally, this post-translational modification of Abeta is more neurotoxic than other Abeta counterparts.

Collectively, these data suggest the critical role of QC in the neuropathology of AD. The present inventors postulated that if an imaging probe of QC was available, it would help to assess QC activity in-vivo, both in preclinical and clinical settings. This probe would serve not only for AD detection, but also for staging and imaging response to clinical therapy (FIG. 1).

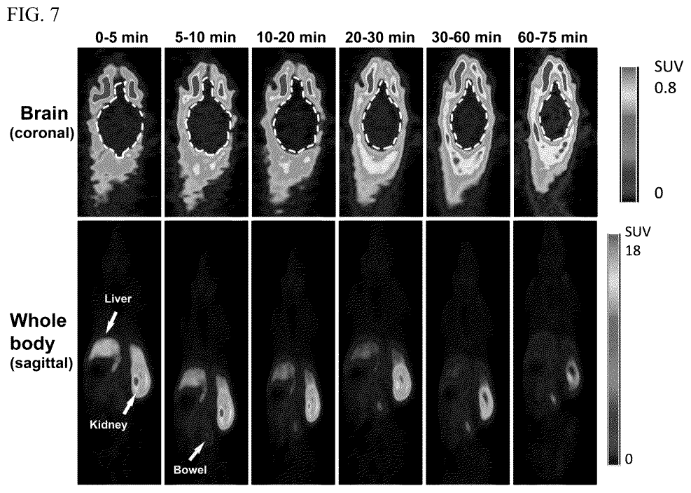

The presently-disclosed subject matter includes compounds of the formula (I); kits for converting a synthetic precursor of a compound of Formula I including a detectable halogen group to a compound of Formula I including a detectable halogen group; pharmaceutical compositions comprising such compounds; methods for preparing compounds of Formula I having a detectable halogen group; and imaging methods and methods for monitoring the severity of a disease or disorder associated with the presence of amyloid in a subject, each by administering to the subject an effective amount of the compound of Formula I comprising a detectable halogen group. In some aspects, the presently-disclosed subject matter includes structurally-unique compounds for imaging QC activity in disease states associated with the up-regulation (e.g., overexpression) of QC. Up-regulation of QC has been observed in disease states such as AD, Parkinson's Disease. Amyotrophic Lateral Sclerosis (ALS). Frontotemporal Dementia (FLD), mild cognitive impairment, Familial British Dementia, Familial Danish Dementia, neurodegeneration in Down Syndrome and Huntington's disease. Recent experiments suggest that QC may be over-expressed in tissues other than the central nervous system (CNS). For example, QC is also present in several peripheral tissues, including the liver, kidneys, heart, and gastrointestinal system, suggesting that it has a broader role in post-translational protein modifications throughout the body. In particular, it was demonstrated in the Examples of this application that there is strong QC activity in the kidneys (see FIG. 7). Indeed, as announced by Vivoryon Therapeutics in April 2024, although varoglutamstat had not changed cognition or function in AD patients, going forward, it will be used to treat kidney disease, based on the results of the clinical trials. Furthermore, presently-disclosed subject matter may expedite the screening of QC inhibitors and evaluate the efficacy of these drugs in clinical trials. Since QC is involved in the early onset of AD, detectable compounds of Formula I can potentially help to stratify AD patients admitted to clinical trials; thus, it could hold considerable benefits for future AD diagnosis, prognosis, management, and treatment. The presently-disclosed compounds of Formula I include compounds that are imaging agents and synthetic precursors of compounds that are imaging agents. Gamma imaging, particularly with PET or SPECT, is a valuable tool for detecting amyloid plaques in the brain, a hallmark feature of AD. By using radiotracers that specifically bind to amyloid, clinicians can non-invasively assess the amyloid burden, aiding in the diagnosis, differentiation, and monitoring of AD and other diseases associated with amyloid plaque. The technique is rapidly evolving and holds promise for improving early diagnosis and providing insights into disease progression and treatment efficacy. The sensitivity of PET imaging has great implications in neuroimaging. Not only has it enabled the use of radioligands with low doses, at sub-pharmacological levels, but it also accommodates probes that have low-to-moderate bioavailability in the brain, which would be otherwise impossible to realize with other imaging modalities. ¹¹C and ¹⁸F, which emit positrons, are preferred for clinical imaging, especially in PET and SPECT techniques. Both ¹¹C and ¹⁸F have very short half-lives (i.e., 20 minutes and 110 minutes, respectively). Therefore, the labeled compounds must be used immediately after preparation. The preferred commercial isotope used for PET applications is ¹⁸F. However, owing to the short half-life, ¹⁸F is particularly demanding when it comes to the preparation of imaging agents. This isotope does not allow for complicated long synthesis routes and purification procedures, since otherwise a considerable amount of the radioactivity of the isotope will already have decayed before the labeled compound can be used for imaging.

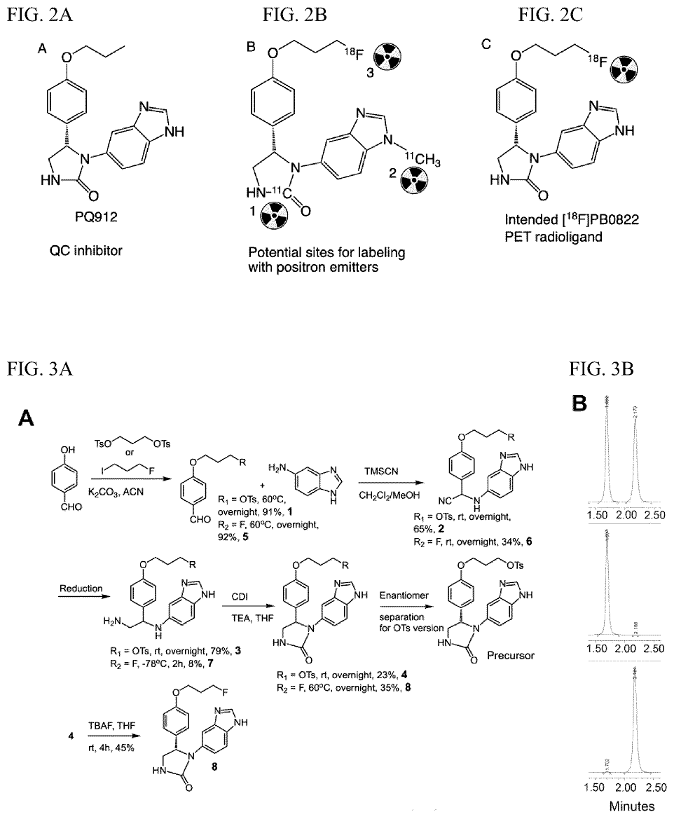

Three potential positions exist to mimic PQ912 as a positron emitter (FIG. 2). Direct [¹¹C] carboxylation using [¹¹C]CO₂ as a synthon could be achieved by directly forming the cyclic urea. Several methods have been developed and reported in the past for synthesizing ureas from CO₂ (see Fuchter, M. J et al. Clean and efficient synthesis of O-silylcarbamates and ureas in supercritical carbon dioxide. Chem Commun (Camb) 2008, (18), 2152-2154; Peterson, S. L. et al. Parallel synthesis of ureas and carbamates from amines and CO2 under mild conditions. Org Lett 2010, 12 (6), 1340-1343; Rotstein, B. H. et al. 11CO2 fixation: a renaissance in PET radiochemistry. Chem Commun (Camb) 2013, 49 (50), 5621-5629; and Wang, H. et al. Synthesis of Ureas from CO2. Top Curr Chem (Cham) 2017, 375 (2), 49). This knowledge has been translated into the successful synthesis of [¹¹C]PQ912 via the incorporation of [¹¹C]CO₂ (see U.S. Pat. No. 8,945,510). Another labeling route disclosed in U.S. Pat. No. 8,945,510 is the [¹¹C]methylation of the amino group on the imidazole ring. As much as chemists may appreciate the convenience of the intrinsic incorporation of the radioisotopes and retaining the same biological activity, the short half-life of [¹¹C] carbon impedes robust in-vivo application. Further, the short half-life of [¹¹C]-probes may be impractical due to the compensatory high radioactivity exposure to chemists as well as testing subjects. Thus, the present inventors developed compounds wherein the radiolabel is incorporated as a substituent instead of as a ring-forming member of the core of Formula I (e.g., ¹⁸F) to overcome these issues. In particular, the radiolabel was incorporated into positions other than those corresponding to ring-forming variables B₁, B₂, B₃, C₁, C₂, C₃, X₁, X₂, Y₁, Y₂, Z, or a ring-forming member of ring A.

The compounds of Formula I have the following formula

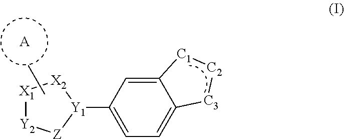

wherein (i) the compound is an imaging agent and at least one substituent comprises a detectable halogen group; or (ii) the compound is a synthetic precursor of an imaging agent, wherein at least one substituent comprises a detectable halogen group.

In Formula I,

A is a 5- or 6-membered aryl ring, or a 5- or 6-membered heteroaryl ring;
a is 0 to 5;
a bond between X₁ and X₂ is a single bond, and X₁ and X₂ are each independently O, S, C(Rₓ), C(Rₓ)₂, —C(Rₓ)₂—C(Rₓ)₂—, —C(Rₓ)₂—O—, or —C(Rₓ)═C(Rₓ)—, wherein at least one of X₁ and X₂ is not —C(Rₓ)₂—C(Rₓ)₂— or —C(Rₓ)₂—O—; or
a bond between X₁ and X₂ is a double bond, and X₁ and X₂ are each independently C or C(Rₓ);
Y₁ is CH, CH(R_{y}), or N;
Y₂ is CH₂, CH(R_{y}), C(R_{y})₂, O, S, NH, or NR₁₀;
Z is C(R_{z})₂ or Z′(═X′)_{x′}, wherein x′ is 1 or 2;
Z′(═X′)_{x′} is C(═O), C(═CH(R_{x′})), C(═C(R_{x′})₂), C(═S), S(═O), or S(═O)₂;
B₁, B₂, and B₃ are each independently CH, C(R_{b}), or N;
C₁, C₂, and C₃ are each independently CH, C(R_{c}), or N;
each occurrence of Rₓ is independently hydrogen or a substituent;
each occurrence of Rₐ, R_{b}, R_{c}, R₁₀, R_{y}, and R_{z} is independently a substituent.

The substituents of Formula I may include:
hydroxyl, deuterated hydroxyl, thiol, deuterated thiol, cyano, halogen, isonitrile (—NC), tri C₁-C₁₂ alkylammonium, C₁-C₁₂ alkyl ketone, sulfonate, or nitro; or
a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), optionally substituted with a deuterium, hydroxyl, deuterated hydroxyl, thiol, deuterated thiol, cyano, halogen, isonitrile (—NC), tri C₁-C₁₂ alkylammonium, C₁-C₁₂ alkyl ketone, sulfonate, or nitro, or a combination thereof,
wherein any carbon-carbon single bond of the C₁-C₁₂ alkyl and the C₁-C₆ alkyl is optionally replaced by at least one carbon-carbon double or triple bond, and any methylene of the C₁-C₁₂ alkyl and the C₁-C₆ alkyl is optionally replaced by at least one O, S, NR₁₀, oxo (—C═O), imido (—C═NR₁₀), thioxo (—C═S), sulfoxo (S═O), sulfone (S(═O)₂), Se, Ge, or Si.

In some aspects, a substituent may include: one or more substituents, wherein each occurrence of a substituent is independently T′ or V₁-T′, wherein: T′ is hydroxyl (—OH), thiol (—SH), cyano (—CN), fluorine (—F), isonitrile (—NC), nitro (—NO₂), —N═O(V₂), NH₂, —NH—V₂, —N(V₂)₂, —O—V₂, —S—V₂, —S(═O)(V₂), —S(═O)(V₂)(NV₂), —S(═O)₂(V₂), aldehyde (—CH(═O), —C(═O)V₂, —C(═S)V₂, —O—C(═O)V₂, —O—C(═S)V₂, —S—C(═O)V₂, —S—C(═S)V₂, —C(═O)—OV₂, —C(═S)V₂, —C(═S)—OV₂, —C(═O)—SV₂, —C(═S)—SV₂, —O—C(═O)—OV₂, —O—C(═S)—OV₂, —O—C(═O)—SV₂, —O—C(═S)—SV₂, —S—C(═O)—OV₂, —S—C(═S)—OV₂, —S—C(═O)—SV₂, —S—C(═S)—SV₂, —C(═O)—NH₂, —C(═O)—NH(V₂), —C(═O)—N(V₂), —C(═S)—NH₂, —C(═S)—NH(V₂), —C(═S)—N(V₂)₂, —NH—C(═O)—V₂, —N(V₂)—C(═O)—V₂, —NH—C(═S)—V₂, —N(V₂)—C(═S)—V₂, NH—S(═O)₂—V₂, N(V₂)—(═O)₂—V₂, —O—C(═O)—NH₂, —O—C(═O)—NH(V₂), —O—C(═O)—N(V₂)₂, —O—C(═S)—NH₂, —O—C(═S)—NH(V₂), —O—C(═S)—N(V₂)₂, —NH—C(═O)—NH₂, —NH—C(═O)—NH(V₂), —NH—C(═O)—N(V₂), —NH—C(═S)—NH₂, —NH—C(═S)—NH(V₂), —O—C(═S)—N(V₂)₂, —NH(V₂)—C(═O)—NH₂, —NH(V₂)—C(═O)—NH(V₂), —NH(V₂)—C(═O)—N(V₂)₂, —NH(V₂)—C(═S)—NH₂, —NH(V₂)—C(═S)—NH(V₂), —O—C(═S)—N(V₂)₂, carboxylic acid (C(═O)OH), sulfonic acid (—SO₃H), —C(═O)NH—NH₂, —C(═S)NH—NH₂, —S(═O)NH—NH₂, —C(═O)NH—NH₂, —C(═S)NH—NH₂, or —C(═NH—NH₂)V₂; each occurrence of V₁ and each occurrence of V₂ independently comprise C₁-C₁₂ alkyl, C₂-C₁₂ alkenyl, C₂-C₁₂ alkynyl, C₃-C₆ cycloalkyl, C₁-C₆ alkyl(C₃-C₆ cycloalkyl), C₁-C₆ alkyl(C₃-C₆ cycloalkenyl), C₃-C₆ cycloalkenyl, C₂-C₆ heterocycloalkyl, C₂-C₆ heterocycloalkenyl, C₁-C₆ alkyl(C₂-C₆ heterocycloalkyl). C₁-C₆ alkyl(C₂-C₆ heterocycloalkenyl), C₅-C₁₂ aryl, C₂-C₁₂ heteroaryl, C₁-C₆ alkyl (C₂-C₁₂ heteroaryl), wherein each substituent is optionally substituted with a deuterium, hydroxyl, deuterated hydroxyl, thiol, deuterated thiol, cyano, halogen, isonitrile (—NC), tri C₁-C₁₂ alkylammonium, C₁-C₁₂ alkyl ketone, sulfonate, or nitro, or a combination thereof.

In Formula I, the “A” ring is a 5- or 6-membered aryl ring, or a 5- or 6-membered heteroaryl ring. As used herein. “aryl ring” refers to an aromatic ring wherein all ring members are carbon and “heteroaryl ring” refers to an aromatic ring wherein at least one of the ring members is a heteroatom. In some aspects. “A” is a 6-membered aryl or a 6-membered heteroaryl ring. In some aspects. “A” includes a benzene ring, a pyridine ring, or a pyrimidine ring, each optionally substituted with Rₐ. In a preferred aspect. “A” is a benzene ring, optionally substituted with Rₐ.

The compounds of Formula I including a detectable halogen group may have a clogP from about 2.0 to about 3.5. When the compound of Formula I is an imaging agent, then the compound may have a clogP from about 2.0 to about 3.5 and the compound may have at least 95% radiochemical purity as determined by analytical HPLC, equipped with a UV absorption detector and a radioisotope detector.

The compounds of Formula (1) may be imaging agents including a detectable halogen group. As used herein, a “detectable halogen group” includes a radiolabel, i.e., a compound where one or more atoms are replaced or substituted by an atom having an atomic mass or mass number different from the atomic mass or mass number typically found in nature (i.e., naturally occurring). Exemplary detectable halogen groups include ¹⁸F, ¹²⁰I, ¹²³I, ¹³¹I, ⁷⁵Br, and ⁷⁶Br. One or more detectable halogen groups may be present. In some aspects, one detectable halogen group is present. In a preferred aspect, the detectable halogen group is ¹⁸F. Advantageously, the presently-disclosed radiolabeled compounds of Formula I may have high radiochemical purity and high molar activity. “Radiochemical purity” refers to the percentage of a radiolabeled compound that consists of the desired radiolabeled compound, as opposed to any impurities and by-products that may have formed during the synthesis process. Radiochemical purity directly impacts the quality and accuracy of the imaging results. If the radiopharmaceutical is not pure, the image might reflect unwanted signals from impurities, leading to false positives, false negatives, or misinterpretation of the results. For example, if a PET tracer designed to bind to specific targets in the brain or a tumor contains a large fraction of impurities that do not bind to the intended target, it can lead to incorrect localization of the tracer, affecting diagnosis and treatment planning. A high radiochemical purity ensures that the majority of the radioactivity is localized in the tissues or areas of interest (e.g., brain), minimizing unnecessary radiation exposure to healthy tissues. The compounds of Formula I may have greater than 95%, greater than 96%, greater than 97%, greater than 98%, at least 99%, at least 99.1%, at least 99.2%, at least 99.3%, at least 99.4%, at least 99.5%, at least 99.6%, at least 99.7%, at least 99.8%, at least 99.9%, or greater than 99.9% radiochemical purity as determined by high-pressure liquid chromatography (HPLC). This ensures that more than 95% of the radioactivity in the formulation is associated with the desired radiolabeled compound and that any impurities are present in amounts below the threshold for clinical use.

“Molar activity” of a radiolabeled compound refers to the amount of radioactivity per mole of the radiolabeled compound. Molar activity is the ratio of radioactivity (usually measured in becquerels (Bq) or curies (Ci)) to the amount of the radiolabeled compound in moles. It is typically expressed in units like MBq/μmol (megabecquerels per micromole), GBq/μmol (gigabecquerels per micromole), or Ci/mmol (curies per millimole). High molar activity means that a large amount of radioactivity is present in a small amount of the radiolabeled compound. This is desirable in many cases, as it allows for efficient labeling of targets with minimal interference from non-radioactive isotopes or unlabeled molecules.

In PET and SPECT, a conventional imaging agent is injected intravenously into the patient. The tracer travels through the bloodstream and crosses the blood-brain barrier, binding specifically to amyloid plaques in the brain. After injection, a waiting period (typically 30-60 minutes) is allowed for the imaging agent to be taken up by the brain tissue, specifically by amyloid plaques. In the presently-disclosed Examples, a ¹⁸F radiolabeled compound F-1 was prepared (structure shown below), the S-isomer of compound F.

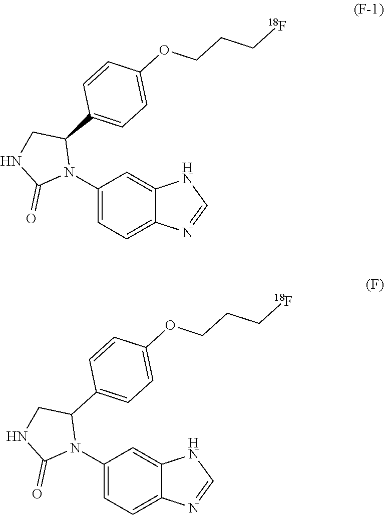 [SMILES: O=C1NC[C@@H](c2ccc(OCCC[18F])cc2)N1c1ccc2nc[nH]c2c1 ; O=C1NCC(c2ccc(OCCC[18F])cc2)N1c1ccc2nc[nH]c2c1]

The radiolabel was introduced in the final step of the synthesis, which is an advantage, given the short half-lives of radioisotopes used in PET and SPECT imaging. Conventional PET imaging agents used for detecting amyloid plaques often suffer from instability, poor yield, or lengthy synthesis processes, which hinder their widespread clinical use. For example, in a conventional approach disclosed in U.S. Pat. No. 8,945,510, the ¹¹C isotope was introduced in the first step of the synthesis, where the second step with formic acid required a 10 minute reaction time, followed by HPLC purification, making such an approach impractical in a clinical setting.

In addition to ease of synthesis, compound F-1 traverses the BBB. In particular, the presently-disclosed Examples disclose that dynamic PET scans obtained immediately after intravenous injection of 5XFAD mice with compound F-1, distribution to the brain 5 minutes post-intravenous injection was accomplished, thus demonstrating that compound F-1 can cross the BBB and pinpoint QC in the brains of 5XFAD mice. 5XFAD mice are genetically engineered to express five familial AD (FAD)-linked mutations in the amyloid precursor protein (APP) and presenilin 1 (PS1) genes, which are associated with the development of early-onset AD in humans.

A factor in determining whether a compound can cross the BBB is its clogP value, which is a measure of its lipophilicity, calculated as the logarithm of the ratio of concentrations of a compound in a hydrophobic (usually octanol) and hydrophilic (usually water) phase. A log P greater than 0 indicates a compound that is more lipophilic, whereas a log P of less than 0 indicates a compound is more hydrophilic. cLogP is an estimate of the partition coefficient, calculated using computational methods, rather than directly measured through experiments. There are several models and algorithms that calculate cLogP, including the Kowwin, Meyers, and Syracuse methods. These algorithms predict cLogP based on the molecular structure of a compound (e.g., its functional groups, molecular weight, and the presence of polar or non-polar regions). A cLogP in the range of 2.0-3.5 is often considered suitable for BBB penetration. The early accumulation and retention of compound F-1, having a cLogP of less than 2, in the brains of 5XFAD mice was modest, yet abundant enough for detection. In some aspects, the compounds of Formula I have a clogP of greater than at least 2.0, at least 2.1, at least 2.2, at least 2.3, at least 2.4, at least 2.5, at least 2.6, at least 2.7, at least 2.8, at least 2.9, at least 3.0, and not more than 3.5.

Another measure of a compound's ability to cross the BBB and reach the brain parenchyma is the standard uptake value (SUV). The SUV is a measurement commonly used in PET imaging to quantify the relative concentration of a radiotracer within a specific tissue or region of interest (ROI), such as in the brain during amyloid PET imaging. It provides a standardized way to assess the metabolism or distribution of the radiotracer in the body. The SUV is calculated using the following formula: tissue radioactivity concentration divided by injected dose/body weight. The tissue radioactivity concentration is the amount of radiotracer in a given tissue (measured in units like kBq/mL or Bq/mL). The injected dose is the total amount of radiotracer injected into the patient (measured in units like MBq or mCi). The SUV is dimensionless, meaning it does not have units. It allows for the comparison of radiotracer uptake between patients of different sizes and body compositions. A SUV in the range of 1.0-1.5 is often considered suitable for BBB penetration. In some aspects, the compounds of Formula I have a SUV of 1.0-1.5.

In some aspects, the compounds of Formula I include compound F and F-1.

The compounds of Formula I may be synthetic precursors to the imaging agents of Formula I. The ¹⁸F labeled compounds of Formula I may be prepared using nucleophilic aromatic substitution and nucleophilic aliphatic substitution. ¹⁸F, having a half-life of ¹¹O minutes must be prepared on-site. ¹⁸F is produced with a cyclotron primarily by proton (¹H) irradiation of ¹⁸O, a stable naturally occurring isotope of oxygen. When the target is liquid H₂¹⁸O, an aqueous solution of ¹⁸F-fluoride ion is obtained; when the target is ¹⁸O₂ gas, ¹⁸F—F₂ gas is obtained. ¹⁸F—F₂ is also prepared from deuteron-irradiation of Ne. The production method used is dependent on the desired subsequent chemical reactions. ¹⁸F-fluoride is produced for use as a nucleophile, while ¹⁸F-fluorine is produced for use in electrophilic methods. Because ¹⁸F-fluoride produced for use as a nucleophile has a much higher specific activity than ¹⁸F-fluorine produced using electrophilic methods, ¹⁸F-fluoride is predominantly used in nuclear medicine. To achieve nucleophilic fluorination, the ¹⁸F-fluoride must be substantially dehydrated by evaporation of the water and subsequent displacement reactions conducted in polar aprotic organic solvents. The solubility and nucleophilicity of fluoride ion may be enhanced by the addition of a phase transfer catalyst (PTC) (such that the cryptand 1,4,7,10-Tetraazacyclododecane-N,N′,N″,N′″-tetraacetic acid, referred to as Kryptofix₂₂₂, which complexes potassium) or by the addition of bulky tetrabutylammonium cation. Radiofluorinations are typically conducted in the presence of poorly nucleophilic bases (e.g., carbonate or bicarbonate ions). Thus, the aqueous solution of ¹⁸F-fluoride obtained from the cyclotron target is treated with the desired salt (cation and, if necessary, a complexing agent, and desired anion) prior to evaporation of the water, which may be accomplished by the azeotropic distillation of water using acetonitrile (CH₃CN).

A wide range of precursors, leaving groups, and reaction conditions can be utilized for ¹⁸F-fluoride nucleophilic substitution. Aliphatic nucleophilic fluorination involves the S_{N}2 substitution of ¹⁸F-fluoride into precursors that contain a leaving group (see Zhang, M. R. and Suzuki, K. (2007) [18F]Fluoroalkyl agents: synthesis, reactivity and application for development of PET ligands in molecular imaging Curr. Top. Med. Chem. 7, 1817-1828; Pretze, M., et al. (2010) The traceless Staudinger ligation with fluorine-18: a novel and versatile labeling technique for the synthesis of PET-radiotracers Tetrahedron Lett. 51, 6410-6414; Zhou, D., et al. (2009) [18F]- and [11C]-Labeled N-benzyl-isatin sulfonamide analogues as PET tracers for apoptosis: synthesis, radiolabeling mechanism, and in vivo imaging study of apoptosis in Fas-treated mice using [11C]WC-98 Org. Biomol. Chem. 7, 1337-1348).

¹⁸F nucleophilic aromatic substitution of an aryl ring requires sufficient activation of the phenyl ring, which can be achieved with electron withdrawing group(s) (e.g., —NO₂, —CN, —CF₃, or carbonyl groups) in the ortho or para position to the leaving group. Aromatic nucleophilic substitution is conducted in a polar aprotic solvent and requires higher temperatures than aliphatic substitution (typically above 100° C.). Aromatic exchange of ¹⁹F by ¹⁸F is feasible in the presence of carbonate and Kyrptofix₂₂₂ or anhydrous tetrabutylammonium fluoride (TBAF) in DMSO, but is not preferred because it results in a lower specific activity radiotracer as compared with other methods. The ¹⁸F labeled compounds may be prepared by nucleophilic aromatic substitutions of an trimethylammonium salt or nitro group. Lower temperatures (100-110° C.) are normally used for an aromatic fluorination on trimethylammonium group compared with a nitro group at elevated temperatures (Tredwell, M. and Gouverneur, V. (2012) 18F labeling of arenes Angew. Chem., Int. Ed. Engl. 51, 11426-11437). Acetonitrile is often used as solvent in a closed reactor system for trimethylammonium displacement. Because of the higher temperatures required for substitution of nitro group, DMF or DMSO are used as solvent.

Heteroaryl groups containing a nitrogen are more electron deficient than the corresponding aromatic hydrocarbon and, thus, are amenable to direct substitution for ¹⁸F-fluoride without an additional activating group as described above (see Dolle, F. (2005) Fluorine-18-labelled fluoropyridines: advances in radiopharmaceutical design Curr. Pharm. Des 11, 3221-3235). For example, the substituted pyridine allows direct ¹⁸F substitution with high radiochemical yields, using NO₂, N⁺Me₃, Br, I, or Cl as a leaving group at 2- and 4-positions. 18F-labeling on heteroaryl groups is conducted in the presence of potassium carbonate and Kyrptofix₂₂₂ with DMSO or DMF as solvent at high temperature (120-150° C.). ¹⁸F-radiolabeled compounds may also be prepared using conventional fluorinating agents such as diethylaminosulfur trifluoride (DAST), N-fluorobenzenesulfonimide (NFSI), SelectFluor, and pyridine hydrofluoride that have been radiolabeled with ¹⁸F (see Gouverneur, V. et al. Angew. Chem. Int Ed. 2012, 51, 2-14).

The conversion of the synthetic precursor of Formula I to an imaging agent of Formula I including a detectable halogen group should proceed with good radiochemical yield (RCY). Radiochemical yield is a measure of how much of the desired radiolabeled compound is obtained relative to the starting material. It is important in the synthesis of radiopharmaceuticals for PET and SPECT imaging, where high yields ensure adequate quantities of the product for use in medical imaging and diagnostics. In some aspects, the conversion of synthetic precursor to imaging agent is accomplished using a nucleophilic substitution reaction, wherein a leaving group is displaced by ¹⁸F. Exemplary leaving groups include sulfonates, for example, mesylates, tosylates, triflates, nosylates, and the like.

The compound of Formula I may be represented by one of Formulas IIA-IIB.

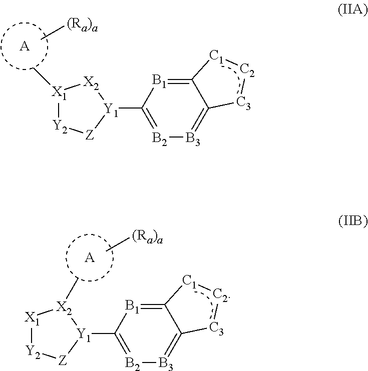

In some aspects of Formulas I, IIA, and IIB, the bond between X₁ and X₂ is a double bond. In some aspects of Formulas I, IIA, and IIB, Z is Z′(═X′)_{x′}; and Y₁ is N; or Y₂ is NH or NR₁₀. In some aspects of Formulas I, IIA, and IIB, Z is Z′(═X′)_{x′}, wherein Z′(═X′)_{x′} is C(═CH(R_{x′})), C(═C(R_{x′})₂), S(═O), or S(═O)₂. In some aspects of Formulas I, IIA, and IIB, Z is Z′(═X′)_{x′}, wherein Z′(═X′)_{x′} is S(═O) or S(═O)₂. In some aspects of Formulas I, IIA, and IIB, B₁, B₂, and B₃ are each independently CH or C(R_{b}), and C₁, C₂, and C₃ are each independently CH or C(R_{c}).

The compound of Formula I may be represented by one of Formulas IIIA-1, IIIA-2, IIIB-1, and IIIB-2.

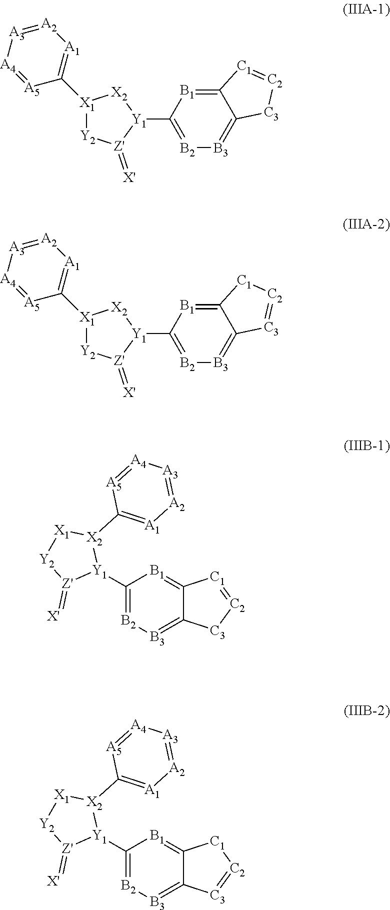

wherein A₁-A₅ are each independently CH, C(Rₐ), or N.

The compound of Formula I may be represented by one of Formulas IVA, IVB, IVC, and IVD:

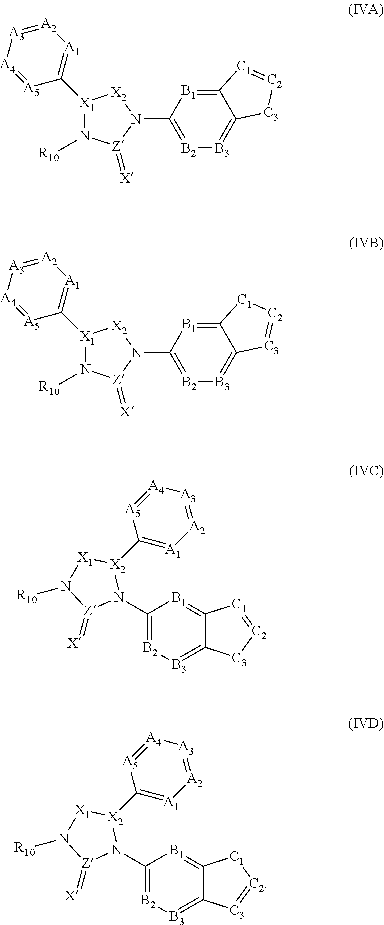

The compound of Formula I may be represented by one of Formulas VA, VB, VC, and VD:

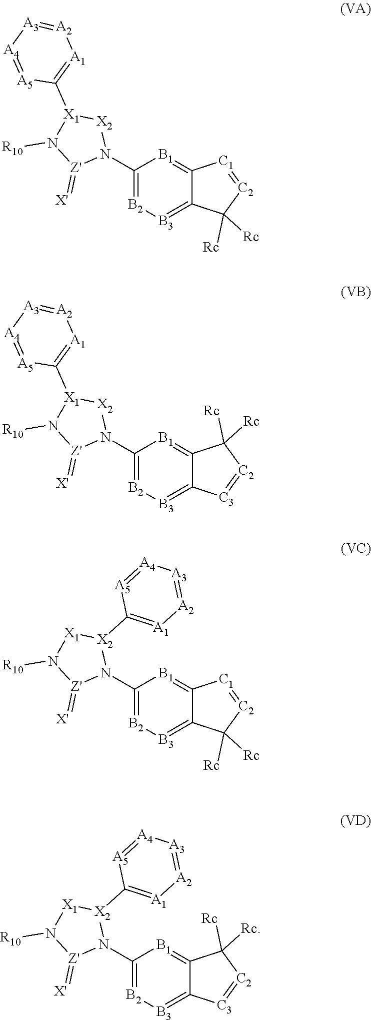

In Formulas I, IIA, IIB, IIIA-1, IIIA-2, IIIB-1, IIIB-2, IVA-IVD, and VA-VD, the compound is a synthetic precursor and at least one of Rₐ, R_{b}, or R_{c} is present and comprises a leaving group. In some aspects, at least one of Rₐ, R_{b}, or R_{c} is present and comprises a sulfonate group, nitro, trialkyl ammonium salt, Br, Cl, I, ¹⁹F, CN, CF₃, or ketone (C(═O)alkyl): a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), each optionally substituted with a deuterium, a halogen, or a combination thereof, wherein any methylene is optionally replaced by at least one O, S, NR₁₀, oxo (—C═O), imido (—C═NR₁₀), thioxo (—C═S), sulfoxo (S═O), sulfone (S(═O)₂), Se, Ge, or Si. In some aspects, at least one of Rₐ, R_{b}, or R_{c} is present and comprises a sulfonate group, nitro, trialkyl ammonium salt, Br, Cl, I, ¹⁹F, CN, CF₃, or ketone (C(═O)alkyl): a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₅-C₁₂ aryl, a C₁-C₆ alkyl (C₅-C₁₂ aryl), optionally substituted with at least one deuterium, a sulfonate group, nitro, trialkyl ammonium salt, Br, Cl, I, ¹⁹F, CN, CF₃, or ketone (C(═O)alkyl).

In some aspects, at least one of Rₐ, R_{b}, or R_{c} is present and comprises one of the following groups comprising a detectable halogen group, preferably ¹⁸F: a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), each optionally substituted with a deuterium, a halogen (other than ¹⁸F), or a combination thereof, wherein any methylene is optionally replaced by at least one O, S, NR₁₀, oxo (—C═O), imido (—C═NR₁₀), thioxo (—C═S), sulfoxo (S═O), sulfone (S(═O)₂), Se, Ge, or Si. In some aspects, at least one of Rₐ, R_{b}, or R_{c} is present and comprises one of the following groups comprising a detectable halogen group, preferably 18F: a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₅-C₁₂ aryl, a C₁-C₆ alkyl (C₅-C₁₂ aryl), optionally substituted with at least one deuterium.

In Formulas I, IIA, IIB, IIIA-1, IIIA-2, IIIB-1, IIIB-2, IVA-IVD, and VA-VD, at least one of the conditions i)-iii) may be true: i) at least one of A₁-A₅ comprises C(Rₐ), wherein Rₐ is a substituent is T′ or V₁-T′; ii) at least one of B₁-B₃ comprises C(R_{b}), wherein R_{b} is a substituent is T′ or V₁-T′; iii) at least one of C₁-C₃ comprises C(R_{c}), wherein Re is a substituent is T′ or V₁-T′; wherein the at least one Rₐ of condition i), or the at least one R_{b} of condition ii), or the at least one R_{c} of condition iii), or a combination thereof is a substituent having formula T′ or V₁-T′. Groups T′ and V₁-T′ are as described in relation to Formula I.

In Formulas I, IIA, IIB, IIIA-1, IIIA-2, IIIB-1, IIIB-2, IVA-IVD, and VA-VD, at least one of the conditions i)-iii) may be true: i) at least one of A₁-A₅ comprises C(Rₐ), wherein Rₐ comprises formula -Lₐ-W; ii) at least one of B₁-B₃ comprises C(R_{b}), wherein R_{b} comprises formula -Lₐ-W; iii) at least one of C₁-C₃ comprises C(R_{c}), wherein R_{c} comprises formula -Lₐ-W; wherein the at least one Rₐ of condition i), or the at least one R_{b} of condition ii), or the at least one R_{c} of condition iii), or a combination thereof comprises -Lₐ-W. -Lₐ-W is not limited to compounds where W is a terminal group on the Lₐ. The W group can be present at any position on the Lₐ group. In formula -Lₐ-W, Lₐ is: a single bond, Lₐ₁, or Lₐ₂. W is a sulfonate, cyano, halogen, isonitrile (—NC), tri C₁-C₁₂ alkylammonium, C₁-C₁₂ alkyl ketone, or nitro, preferably a sulfonate; or a detectable halogen group, preferably ¹⁸F.

Lₐ₁ comprises a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), optionally substituted with a deuterium, a halogen, or a combination thereof, wherein any methylene is optionally replaced by at least one O, S, NR₁₀, oxo (—C═O), imido (—C═NR₁₀), thioxo (—C═S), sulfoxo (S═O), sulfone (S(═O)₂), Se, Ge, or Si; and W is a sulfonate, cyano, halogen, isonitrile (—NC), tri C₁-C₁₂ alkylammonium, C₁-C₁₂ alkyl ketone, or nitro, preferably a sulfonate; or a detectable halogen group, preferably ¹⁸F. Lₐ₂ is represented by formula U-L_{a2′}, wherein U is O or S, and L_{a2′} comprises a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), optionally substituted with a deuterium, a halogen, or a combination thereof. In some aspects, Lₐ₁ and L_{a2′} each independently comprise a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₅-C₁₂ aryl, a C₁-C₆ alkyl (C₅-C₁₂ aryl), each optionally substituted with a deuterium, a halogen, or a combination thereof. W is a sulfonate, cyano, halogen, isonitrile (—NC), tri C₁-C₁₂ alkylammonium, C₁-C₁₂ alkyl ketone, or nitro, preferably a sulfonate; or a detectable halogen group, preferably ¹⁸F.

The details of one or more aspects of the presently-disclosed subject matter are set forth in this document. Modifications to aspects described in this document, and other aspects, will be evident to those of ordinary skill in the art after a study of the information provided in this document. The information provided in this document, and particularly the specific details of the described exemplary aspects, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims, unless the context clearly dictates otherwise.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some aspects ±20%, in some aspects ±10%, in some aspects ±5%, in some aspects ±1%, in some aspects ±0.5%, and in some aspects ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Embodiments of the present invention include pharmaceutical compositions comprising a radiolabeled compound as described herein or a pharmaceutically acceptable salt, solvate or polymorph thereof, including all tautomers and stereoisomers thereof, in combination with one or more pharmaceutically acceptable excipients. In some aspects, the pharmaceutical composition is for use as an imaging agent in the detection of a neurological disorder. In some aspects, the pharmaceutical composition is for use as an imaging agent in the detection of a neurological disorder selected from mild cognitive impairment, Alzheimer's disease, Familial British Dementia, Familial Danish Dementia, neurodegeneration in Down Syndrome and Huntington's disease, such as Alzheimer's disease.

In some aspects, the radiolabeled compound or pharmaceutical composition is for use in the detection of amyloid peptides. In some aspects, the radiolabeled compound or pharmaceutical composition is for use in the detection of tau proteins of neurofibrillary tangles.

The present teachings include a method for imaging and detection of senile plaques and/or neurofibrillary tangles in a brain tissue, the method comprising treating the tissue with an radiolabeled inhibitor compound as described herein for detection of neurological disorders. In some aspects, the neurological disorder is detected by measuring the affinity of an inhibitor as described herein for senile plaques. In some aspects, the neurological disorder is detected by measuring the affinity of an inhibitor as described herein for tau aggregates.

The present teachings include a method for ex vivo or in vitro detection of amyloid deposits in a brain tissue, the method comprising treating the tissue with a radiolabeled inhibitor compound as described herein for detection of the amyloid deposit.

The present teachings include a method for in vivo detection of amyloid deposits in a patient, the method comprising administering an effective amount of a radiolabeled inhibitor compound as described herein to the patient, and detecting the binding level of the compound to the amyloid deposit to the patient.

The present teachings include a method for ex vivo or in vitro detection of tau proteins in a brain tissue, the method comprising treating the tissue with a radiolabeled inhibitor compound as described herein for detection of the neurofibrillary tangles.

The present teachings include a method for in vivo detection of neurofibrillary tangles in a patient, the method comprising administering an effective amount of a radiolabeled inhibitor compound as described herein to the patient, and detecting the binding level of the compound to tau proteins.

In some aspects of the methods described herein, detection is performed using gamma imaging, magnetic resonance imaging, magnetic resonance spectroscopy or fluorescence spectroscopy. In some aspects the detection by gamma imaging is PET or SPECT.

The present invention provides a simplified and efficient PET imaging probe for detecting amyloid plaques, with improved radiochemical purity, stability, and ease of use. The present teachings include a probe for diagnosing a neurological disorder which comprises a pharmaceutical composition as described herein and instructions to use said kit in accordance with the methods described herein. In some aspects of the kit, the neurological disorder is mild cognitive impairment, Alzheimer's disease, Familial British Dementia, Familial Danish Dementia, neurodegeneration in Down Syndrome and Huntington's disease, such as Alzheimer's disease.

The present invention relates to a kit for imaging of amyloid plaques in the brain. The kit contains all necessary components for radiolabeling a precursor compound with [F-18] fluorine, allowing for the preparation of a PET imaging agent with high radiochemical purity and stability. The kit is designed for easy use, with pre-mixed reagents and simple instructions for clinicians to perform the radiolabeling process.

References herein to “radiolabeled” include a compound where one or more atoms are replaced or substituted by an atom having an atomic mass or mass number different from the atomic mass or mass number typically found in nature (i.e., naturally occurring). The term “radiolabeled” may be interchangeably used with “isotopically-labelled”. “labelled”. “isotopic tracer group” “isotopic marker”. “isotopic label”. “detectable isotope” or “radioligand”.

In one aspect of the present invention, the compound of Formula I comprises a single detectable halogen group.

Examples of suitable, detectable halogen groups include: ¹⁸F, ³⁶Cl, ⁸²Br, ⁷⁵Br, ⁷⁶Br, ⁷⁷Br, ¹²³I, ¹²⁴I, ¹²⁵I and ¹³¹I. It is to be understood that an isotopically labeled compound needs only to be enriched with a detectable isotope to, or above, the degree which allows detection with a technique suitable for the particular application. The radionuclide that is incorporated in the radiolabeled compounds will depend on the specific application of that radiolabeled compound. In an aspect, the radiolabel is ¹⁸F.

In view of the close relationship between the free compounds and the compounds in the form of their salts or solvates, whenever a compound is referred to in this context, a corresponding salt, solvate or polymorph is also intended, provided such is possible or appropriate under the circumstances.

Salts and solvates of the compounds of Formula I and physiologically functional derivatives thereof which are suitable for use in medicine are those wherein the counter-ion or associated solvent is pharmaceutically acceptable. However, salts and solvates having non-pharmaceutically acceptable counter-ions or associated solvents are within the scope of the present disclosure, for example, for use as intermediates in the preparation of other compounds and their pharmaceutically acceptable salts and solvates.

Suitable salts according to the present disclosure include those formed with both organic and inorganic acids or bases. Pharmaceutically acceptable acid addition salts include those formed from hydrochloric, hydrobromic, sulfuric, nitric, citric, tartaric, phosphoric, lactic, pyruvic, acetic, trifluoroacetic, triphenylacetic, sulfamic, sulfanilic, succinic, oxalic, fumaric, maleic, malic, mandelic, glutamic, aspartic, oxaloacetic, methanesulfonic, ethanesulfonic, arylsulfonic (for example p-toluenesulfonic, benzenesulfonic, naphthalenesulfonic or naphthalenedisulfonic), salicylic, glutaric, gluconic, tricarballylic, cinnamic, substituted cinnamic (for example, phenyl, methyl, methoxy or halo substituted cinnamic, including 4-methyl and 4-methoxycinnamic acid), ascorbic, oleic, naphthoic, hydroxynaphthoic (for example 1- or 3-hydroxy-2-naphthoic), naphthaleneacrylic (for example naphthalene-2-acrylic), benzoic, 4-methoxybenzoic, 2- or 4-hydroxybenzoic, 4-chlorobenzoic, 4-phenylbenzoic, benzeneacrylic (for example 1,4-benzenediacrylic), isethionic acids, perchloric, propionic, glycolic, hydroxyethanesulfonic, pamoic, cyclohexanesulfamic, salicylic, saccharinic and trifluoroacetic acid. Pharmaceutically acceptable base salts include ammonium salts, alkali metal salts such as those of sodium and potassium, alkaline earth metal salts such as those of calcium and magnesium and salts with organic bases such as dicyclohexylamine and N-methyl-D-glucamine.

All pharmaceutically acceptable acid addition salt forms of the compounds described herein are intended to be embraced by the scope of the present disclosure.

Furthermore, some of the crystalline forms of the compounds may exist as polymorphs and as such are intended to be included in the present disclosure. In addition, some of the compounds may form solvates with water (i.e. hydrates) or common organic solvents, and such solvates are also intended to be encompassed within the scope of this disclosure. The compounds, including their salts, can also be obtained in the form of their hydrates, or include other solvents used for their crystallization.

SPECT imaging with a labeled compound is a non-invasive molecular imaging technique used to visualize specific biological processes in vivo, particularly at the cellular and molecular levels. When compounds of Formula I include a detectable halogen group, they can be tracked in the body using SPECT to identify disease states, such as cancer, inflammation, or neurodegenerative diseases like AD. SPECT imaging detects gamma radiation emitted by the radioactive isotope attached to the compound of Formula I. A gamma camera captures these emissions and constructs images based on the distribution of the radiolabeled compound of Formula I inside the body. SPECT creates 3D images or slices of the subject, which provide valuable information about the location, concentration, and distribution of the radiolabeled compound of Formula I and thus the targeted biomarker. A radioactive isotope (also called a radiolabel) is attached to the compound of Formula I, allowing it to be detected using SPECT imaging.

PET uses positron-emitting radioisotopes to track the distribution of a compound in the body. To enable PET imaging, a radioactive isotope is present in a compound of Formula I, which emits positrons (subatomic particles with a positive charge). Common isotopes used for labeling compounds of Formula I in PET imaging include Fluorine-18 (18F), the most commonly used PET isotope due to its ideal half-life (about 110 minutes) and ease of labeling. It can be incorporated into organic molecules, making it ideal for PET.

According to a further aspect of the present disclosure there is provided a probe for diagnosing a neurological disorder which comprises a pharmaceutical composition as defined herein and instructions to use said kit in accordance with the methods described herein.

The presently-disclosed subject matter includes a pharmaceutical composition comprising the compound of Formula I and a pharmaceutically acceptable carrier. The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed agents. In general, these materials can be formulated in non-toxic, inert, and pharmaceutically acceptable aqueous carrier media wherein the pH is generally about 5-8, preferably about 6-8, although the pH can be varied with the nature of the formulation material and the condition to be treated. The formulated pharmaceutical compositions can be administered by conventional routes including, but not limited to, intratumoral, intraperitoneal, intravenous, or topical administration. The pharmaceutical composition of the present invention contains a safe and effective amount (for example, 0.001-99 wt %, or 0.01-90 wt %, or 0.1-80 wt %) of the above-mentioned compounds of Formula I and pharmaceutically acceptable carriers or excipients. Such carriers include, but are not limited to: saline, buffer, dextrose, water, glycerol, ethanol, and the combinations thereof. The drug formulation should be suitable for the mode of administration. The pharmaceutical composition of the present invention may be prepared in the form of an injection, for example, by a conventional method using physiological saline or an aqueous solution containing glucose and other adjuvant. Pharmaceutical compositions such as injections and solutions are preferably made under aseptic conditions.

The pharmaceutical compositions according to the invention may be administered orally in the form of a suitable pharmaceutical unit dosage form. The pharmaceutical compositions of the disclosure may be prepared in many forms that include tablets, hard or soft capsules, especially hard or soft gelatin capsules, aqueous solutions, suspensions, and liposomes and other slow-release formulations, such as shaped polymeric gels.

The mode of administration and dosage forms are closely related to the properties of the therapeutic agents or compositions which are desirable and efficacious for the given treatment application. Suitable dosage forms include, but are not limited to, oral, intravenous, rectal, sublingual, mucosal, nasal, ophthalmic, subcutaneous, intramuscular, transdermal, spinal, intrathecal, intra-articular, intra-arterial, sub-arachnoid, bronchial, and lymphatic administration, and other dosage forms for systemic delivery of active ingredients.

Pharmaceutical compositions of the disclosure may be administered by any method known in the art, including, without limitation, transdermal (passive via patch, gel, cream, ointment or iontophoretic); intravenous (bolus, infusion); subcutaneous (infusion, depot); transmucosal (buccal and sublingual, e.g., orodispersible tablets, wafers, film, and effervescent formulations; conjunctival (eye drops); rectal (suppository, enema)); or intradermal (bolus, infusion, depot).

The solid unit dosage forms can be of the conventional type. The solid form can be a capsule, such as an ordinary gelatin type containing the inhibitor of the present invention, and optionally an anti-bacterial compound of the present invention, and a carrier, for example, lubricants and inert fillers such as, lactose, sucrose, or cornstarch. In another aspect, these compounds are tableted with conventional tablet bases such as lactose, sucrose, or corn starch in combination with binders like acacia, corn starch, or gelatin, disintegrating agents such as, corn starch, potato starch, or alginic acid, and a lubricant like stearic acid or magnesium stearate.

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

Pharmaceutical compositions of the disclosure may also be formulated for parenteral administration (e.g., by injection, for example, bolus injection or continuous infusion) and may be presented in unit dosage form in ampoules, pre-filled syringes, small volume infusion containers or multi-dose containers with an added preservative. The pharmaceutical compositions may take such forms as suspensions, solutions, or emulsions in oily or aqueous vehicles, and may contain formulating agents such as suspending, stabilizing and/or dispersing agents. Alternatively, the pharmaceutical compositions of the disclosure may be in powder form, obtained by aseptic isolation of sterile solid or by lyophilization from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the pharmaceutical composition with the softened or melted carrier(s) followed by chilling and shaping in molds.

For administration by inhalation, the pharmaceutical compositions according to the invention are conveniently delivered from an insufflator, nebulizer or a pressurized pack or other convenient means of delivering an aerosol spray. Pressurized packs may comprise suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the pharmaceutical compositions of the disclosure may take the form of a dry powder composition, for example, a powder mix of the pharmaceutical composition and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, the pharmaceutical compositions of the disclosure may be administered via a liquid spray, such as via a plastic bottle atomizer. Typical of these are the Mistometerg (isoproterenol inhaler-Wintrop) and the Medihaler®) (isoproterenol inhaler-Riker).

It will be further appreciated that the amount of the pharmaceutical compositions required for use in treatment will vary not only with the therapeutic agent selected but also with the route of administration, the nature of the condition being treated and the age and condition of the patient and will be ultimately at the discretion of the attendant physician or clinician.

The presently-disclosed subject matter includes a method for detecting amyloid (e.g., amyloid plaque), tau proteins of neurofibrillary tangles (e.g., aggregates), or a combination thereof in a subject, the method comprising administering to the subject an effective amount of the compound of Formula (I) comprising a detectable halogen group. The detection of such amyloid peptides has utility in the detection and quantification of amyloid deposits and/or neurofibrillary tangles in diseases include, but are not limited to Mediterranean fever, Muckle Wells syndrome, idiopathetic myeloma, amyloid polyneuropathy, amyloid cardiomyopathy, systemic senile myloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, Isolated atrial amyloid, [beta]2-microglobulin amyloid in dialysis patients, inclusion body myositis. β2-amyloiddeposits in muscle wasting disease, chronic traumatic encephalopathy (CTE), and Islets of Langerhans diabetes Type II insulinoma. Methods include SPECT and PET. An “effective amount” for imaging refers to the quantity that is needed to achieve optimal imaging results while minimizing any potential side effects or toxicity to the subject. The term “effective amount” is context-dependent and varies based on the imaging technique, the agent used, and the biological system being studied. It represents a dose that is sufficient for generating a clear and accurate image without oversaturating or underrepresenting the target. The effective amount of an imaging agent is influenced by its affinity for the target and how well it binds to the target tissue. For highly specific and high-affinity agents, lower doses may be effective for imaging, as these agents can bind to their targets with minimal concentration. In contrast, for agents with lower specificity or affinity, a higher dose may be needed to ensure sufficient target binding and imaging signal. The effective amount for PET imaging involves radiolabeling a with a radioactive isotope. The quantity needed depends on the half-life of the isotope, the target's expression levels, and the size of the area being imaged.

The presently-disclosed subject matter includes a method for monitoring the severity of a disease or disorder associated with protein aggregation in a subject, wherein the method comprises administering to the subject an effective amount of a compound of Formula I comprising a detectable group. The disease or disorder associated with the presence of protein aggregation in a subject may include: mild cognitive impairment, Alzheimer's disease, Familial British Dementia, Familial Danish Dementia, prion disease, multiple system atrophy, spinocerebellar ataxia, cystic fibrosis, alpha-1 antitrypsin deficiency. Wilson's disease, neurodegeneration in Down Syndrome and Huntington's disease. In one particular aspect, the disease or disorder neurological disorder is Alzheimer's disease. In another aspect, the disease or disorder associated with the presence of amyloid in a subject may be a kidney disease.

A disease or disorder associated with protein aggregation in a subject may be amyloidosis. There are several types of amyloidosis, with the most common being AL amyloidosis (Primary Amyloidosis) and AA amyloidosis (Secondary Amyloidosis). There are also other rarer types, including hereditary amyloidosis and senile amyloidosis. In some aspects, the amyloidosis is dialysis-related amyloidosis.

Dose levels can range from about 0.001 μg/kg/day to about 10.000 mg/kg/day. In one aspect, the dose level is about 0.001 μg/kg/day to about 10 g/kg/day. In another aspect, the dose level is about 0.01 μg/kg/day to about 1.0 g/kg/day. In yet another aspect, the dose level is about 0.1 mg/kg/day to about 100 mg/kg/day. The regimen may include pre-treatment and/or co-administration with additional compounds such as for example therapeutic agent(s).

According to a further aspect of the present disclosure there is provided a method for imaging and detection of senile plaques and/or neurofibrillary tangles in a brain tissue, the method comprising treating the tissue with an inhibitor as defined herein for detection of neurological disorders.

One of the key prerequisites for an in vivo imaging agent of the brain is the ability to cross the intact blood-brain barrier after a bolus i.v. injection. In the first step of the present method of imaging, the radiolabeled compound of Formula I of the present disclosure is introduced into a tissue or a patient in a detectable quantity. The compound is typically part of a pharmaceutical composition and is administered to the tissue or the patient by methods well known to those skilled in the art.

In an alternative aspect, the radiolabeled compound of Formula I of the present disclosure is introduced into a patient in a detectable quantity and after sufficient time has passed for the compound to become associated with amyloid deposits and/or tau proteins, the labeled compound is detected non-invasively. In another aspect of the present disclosure, a radiolabeled compound of Formula I described herein is introduced into a patient, sufficient time is allowed for the compound to become associated with amyloid deposits, and then a sample of tissue from the patient is removed and the radiolabeled compound in the tissue is detected apart from the patient. In another aspect of the present disclosure, a tissue sample is removed from a patient and a radiolabeled compound of Formula I of the present disclosure is introduced into the tissue sample. After a sufficient amount of time for the compound to become bound to amyloid deposits and/or tau proteins, the compound is detected.

A detectable quantity is a quantity of labeled compound necessary to be detected by the detection method chosen. The amount of radiolabeled compound of Formula I of the present disclosure to be introduced into a patient in order to provide for detection can readily be determined by those skilled in the art. For example, increasing amounts of the radiolabeled compound can be given to a patient until the compound is detected by the detection method of choice. A label is introduced into the compounds to provide for detection of the compounds.

The amount of time necessary can easily be determined by introducing a detectable amount of radiolabeled compound of Formula I of the present disclosure into a patient and then detecting the radiolabeled compound at various times after administration.

According to a further aspect of the present disclosure there is provided a kit for diagnosing a neurological disorder which comprises a pharmaceutical composition as defined herein and instructions to use said kit in accordance with the methods described herein.

### EXAMPLES

Synthesis of 3-(4-formylphenoxy) propyl 4-methylbenzenesulfonate: Compound 1. To a stirring solution of 4-hydroxy benzaldehyde (1.0 g, 8.2 mmol), and 1,3 propane di-tosylate (6.3 g, 16.4 mmol), in acetonitrile (60 mL) was added K₂CO₃ (2.3 g, 16.4 mmol) and heated to 60° C. overnight. This solution was cooled and diluted with H₂O and EtOAc. The product was extracted 3× with EtOAc. The organic layers were combined, washed with brine, dried over Na₂SO₄, filtered, and concentrated under reduced pressure. The product was purified by flash chromatography (0-100% CH₂Cl₂/(20% MeOH/CH₂Cl₂)) over 18 minutes to provide the final product as a white solid (2.5 g, 91% yield). ¹H CDCl₃ (400.13 mHz): 9.90 (s, 1H); 7.82 (d, J=8.8 Hz, 2H); 7.76 (d, 8.3 Hz, 1H); 7.25 (d, 8.2 Hz, 2H); 6.88 (d, 8.8 Hz, 2H); 4.26 (t, J=5.9 Hz, 2H); 4.05 (t, J=5.9 Hz, 2H); 2.38 (s, 3H); 2.16 (m, 2H); ¹³C CDCl₃ (100.6 mHz): 190.7, 163.4, 144.9, 132.7, 131.9, 130.1, 129.8, 127.8, 114.6, 66.6, 63.4, 28.7, 21.6. HRMS (ESI/Q-TOF) m/z: [M+H]+ Calcd for C17H18O5S, 335.0948; Found 335.0948.

Synthesis of 3-(4-(((1H-benzo[d]imidazol-5-yl)amino)(cyano)methyl)phenoxy)propyl 4-methylbenzenesulfonate: Compound 2. To a stirring solution of 1 (1.0 g, 2.9 mmol) and 2-amino benzimidazole (464 mg, 3.5 mmol) in CH₂Cl₂ (3 mL) and MeOH (3 mL) was added trimethylsilyl cyanide (575 mg, 5.8 mmol). This solution was stirred overnight at room temperature. The reaction was concentrated under reduced pressure. The residue was diluted with H₂O and EtOAc. The product was extracted 3× with EtOAc. The organic layers were combined, washed with brine, dried over Na₂SO₄, filtered, and concentrated under reduced pressure. The product was purified by flash chromatography (0-100% CH₂Cl₂/(20% MeOH/CH₂Cl₂)) over 18 minutes to afford a final product as dark brown solid, (900 mg, 65% yield). Note: to obtain a pure NMR, the sample was purified by flash chromatography. However, the material decomposes while sitting in MeOH, so for higher yields in subsequent steps the reaction was used as is. ¹H MeOD (600.13 mHz): 8.01 (s, 1H); 7.71 (d, J=8.3 Hz, 2H); 7.51 (d, J=8.6 Hz, 2H); 7.45 (d, J=8.7 Hz, 1H); 7.27 (d, J=8.0 Hz, 2H); 7.00 (d, J=2.0 Hz, 1H); 6.87 (dd, J₁=8.7 Hz, J₂=2.2 Hz); 6.84 (d, J=8.7 Hz, 2H); 5.67 (s, 1H), 4.22 (t, J=6.1 Hz, 2H); 3.93 (t, J=5.8 Hz, 2H); 2.32 (s, 3H); 2.08 (m, 2H). ¹³C MeOD (150.9 mHz): 160.5, 146.5, 144.0, 141.4, 134.0, 131.7, 131.1, 129.7, 129.1, 128.9, 128.5, 120.5, 115.9, 115.8, 115.7, 114.4, 68.4, 64.3, 51.0, 29.8, 21.6; HRMS (ESI/Q-TOF) m/z: [M+H]+ Calcd for C25H24N4O4S, 477.1591; Found 477.1588.

Synthesis of 3-(4-(1-((1H-benzo[d]imidazol-5-yl)amino)-2-aminoethyl) phenoxy) propyl 4-methylbenzenesulfonate: Compound 3. To a stirring solution of 2 (900 mg, 1.9 mmol) in acetic acid (15 mL) in a Parr hydrogen bomb was added 10% wet Pd/C (200 mg, 1.9 mmol). The vessel was purged of air in the following way: placed under vacuum and refilled with 130 psi of hydrogen three times. The solution was stirred overnight. Reaction monitoring indicated it was 50% complete, so an addition of equivalent of 10% wet Pd/C (200 mg, 1.9 mmol) was added. The vessel was recharged and purged with 130 psi of hydrogen three times and let stir overnight. Upon completion, the reaction was filtered over celite, washed with MeOH, and concentrated under reduced pressure; acetonitrile was added to help remove the remaining acetic acid. The product was purified by flash chromatography (0-100% CH₂Cl₂/(20% MeOH/CH₂Cl₂)) over 18 minutes to afford a final product as a dark brown solid (720 mg, 79% yield). ¹H DMSO (600.13 mHz): 9.21 (s, 1H); 8.08 (br·s, 3H); 7.72 (d, J=8.3 Hz, 2H); 7.55 (d, J=8.9 Hz, 2H); 7.33 (m, 4H); 6.95 (dd, J₁=8.9 Hz, J₂=2.0 Hz, 1H); 6.80 (d, J=8.8 Hz, 1H); 6.65 (d, J=1.9 Hz, 1H); 4.70 (m, 1H); 4.13 (m, 2H); 3.86 (m, 2H); 3.11 (m, 2H); 2.28 (s, 3H); 1.97 (m, 2H). 13C DMSO (150.9 mHz): 158.7, 158.5, 158.3, 158.0, 157.8, 146.4, 144.9, 138.0, 132.1, 132.0, 131.5, 130.1, 128.1, 127.8, 127.5, 125.5, 122.5, 117.1, 115.7, 115.1, 94.2, 67.6, 63.1, 54.6, 44.4, 40.4, 28.1, 21.0. HRMS (ESI/Q-TOF) m/z: [M+H]+ Calcd for C25H28N4O4S, 481.1904; Found 481.1929.

Synthesis of 3-(4-(3-(1H-benzo[d]imidazol-5-yl)-2-oxoimidazolidin-4-yl)phenoxy)propyl 4-methylbenzenesulfonate: Compound 4. To a stirring solution of 3 (720 mg, 1.5 mmol), triethylamine (1.5 g, 15 mmol) in THF (50 mL) was added 1,1′-Carbonyldiimidazole (243 mg, 1.5 mmol). The solution was stirred at room temperature overnight. This solution was diluted with H₂O and CH₂Cl₂. The product was extracted 3× with CH₂Cl₂. The organic layers were combined, washed with brine, dried over Na₂SO₄, filtered, and concentrated under reduced pressure. The temperature was kept under 30° C. so as to prevent the substitution by imidazole. The product was purified by reverse phase chromatography (Gilson, Phenomenex C18 (00B-4454-DO-AX) Gemini-NX 5u, 110 A, 50×21.20 mm column), 98% H₂O/2% ACN to 100% ACN over 8 minutes to afford the final product as a white solid (170 mg, 23% yield). 1H MeOD (600.13 mHz): 9.05 (s, 1H); 7.85 (s, 1H); 7.66 (d, J=8.28 Hz, 2H); 7.63 (s, 1H); 7.62 (d, J=1.74 Hz, 1H); 7.29 (dd, J₁=6.84 Hz, J₂=6.42 Hz, 2H); 7.20 (d, J=7.98 Hz, 2H); 6.73 (d, J=1.92 Hz, 2H); 5.51 (dd, J₁=9.12 Hz, J₂=6.42 Hz, 1H); 4.14 (m, 2H), 3.98 (t, J=9.12 Hz, 1H); 3.86 (m, 2H); 3.34 (dd, J₁=9.12 Hz, J₂=6.30 Hz, 2H); 2.01 (m, 2H). ¹³C MeOD (150.9 mHz): 162.0, 160.0, 146.3, 141.0, 139.2, 133.4, 132.8, 131.0, 128.94, 128.91, 128.6, 122.0, 116.0, 115.2, 107.5, 68.4, 64.3, 61.8, 48.2, 29.8, 21.5; HRMS (ESI/Q-TOF) m/z: [M+H]+ Calcd for C26H26N4O5S, 507.1697; Found 507.1687.

Synthesis of the standard [¹⁹F]PB0822 (compound 8) starting from tosylate compound 4. To a stirring solution of 4 (15 mg, 0.03 mmol) in 1 mL of THF was added TBAF (1.0 M in THF) (0.200 mL) and was stirred for 4 hours until completion. The resulting solution was concentrated under reduced pressure. The product was purified by reverse phase chromatography (Gilson, Phenomenex C18 (00B-4454-DO-AX) Gemini-NX 5u, 110 A, 50×21.20 mm column), 98% H2O/2% ACN to 60% ACN over 8 minutes to provide a final product with 45% yield.

Synthesis of 4-(3-fluoropropoxy)benzaldehyde: Compound 5. To a stirring solution of 4-hydroxy benzaldehyde (1.0 g, 8.1 mmol) and 1-iodo, 3-fluoro propane (1.54 g, 8.1 mmol) in acetonitrile (50 mL) was added K₂CO3 (1.3 g, 9.7 mmol) and heated to 60° C. overnight. The reaction was concentrated under reduced pressure. The residue was resuspended in CH₂Cl₂ and filtered. The supernatant was concentrated under reduced pressure and purified by flash chromatography. The product was purified by flash chromatography (0-100% CH₂Cl₂/(20% MeOH/CH₂Cl₂)) over 15 minutes to provide the final product as a clear oil (1.36 g, 92% yield). ¹H CDCl₃ (400.13 mHz): 9.87 (s, 1H); 7.82 (d, J=8.0 Hz, 2H); 6.99 (d, J=8.0 Hz, 2H); 4.64 (dt, J₁=48.0 Hz, J₂=5.7 Hz, 2H); 4.17 (t, J=6.1 Hz, 2H); 2.20 ((m, 2H). ¹³C CDCl₃ (100.6 mHz): 190.7, 163.7, 131.9, 130.0, 114.7, 80.3 (d, J=165.0 Hz), 63.8 (d, J=5.0 Hz), 30.2 (d, J=20.1 Hz); HRMS (ESI/Q-TOF) m/z: [M+H]+ Calcd for C10H11FO2 183.0816; Found 183.0795.

Synthesis of 2-((1H-benzo[d]imidazol-5-yl)amino)-2-(4-(3-fluoropropoxy)phenyl) acetonitrile: Compound 6. To a stirring solution of 5 (988 mg, 5.4 mmol) and 2-amino benzimidazole (722 mg, 5.4 mmol) in MeOH (3 mL) was added trimethylsilyl cyanide (642 mg, 6.5 mmol). This solution was stirred overnight at room temperature. The reaction was quenched with saturated bicarbonate and extracted 3× with CH₂Cl₂. The organic layers were combined, washed with brine, dried over Na₂SO₄, filtered, and concentrated under reduced pressure. The product was purified by flash chromatography (0-100% CH₂Cl₂/(20% MeOH/CH₂Cl₂)) over 30 minutes, (598 mg, 34% yield). Note: to obtain a pure NMR, the sample was purified by flash chromatography. However, the material decomposes while sitting in MeOH, so for higher yields in subsequent steps, after reaction completion, it was concentrated and triturated with acetonitrile. The resulting slurry was filtered and used as a dark brown solid. ¹H CDCl₃ (600.13 mHz): 7.95 (s, 1H); 7.58 (br·s, 1H); 7.53 (d, J=5.8 Hz, 2H); 6.99 (m, 3H); 6.76 (dd, J₁=5.8 Hz, J₂=1.5 Hz, 1H); 5.34 (d, J=5.0 Hz, 1H); 4.66 (dd, J₁=31.4 Hz, J₂=3.8 Hz, 2H); 4.13 (t, J=4.0 Hz, 2H); 4.04 (d, J=5.3 Hz, 1H); 2.20 (m, 2H). ¹³C CDCl₃ (150.9 mHz): 159.6, 141.5, 139.8, 130.5, 128.7, 126.1, 118.5, 115.2, 114.7, 112.9, 80.5 (d, J=164.5 Hz), 63.7 (d, J=4.5 Hz), 50.7, 30.3 (d, 19.6 Hz); HRMS (ESI/Q-TOF) m/z: [M+H]+ Calcd for C18H17FN4O, 325.1459; Found 325.1470.

Synthesis of N¹-(1H-benzo[d]imidazol-5-yl)-1-(4-(3-fluoropropoxy)phenyl) ethane-1,2-diamine: Compound 7. A stirring solution of 6 (252 mg, 0.78 mmol) in THF (10 mL) was cooled to −78° C. To this solution was added 1M DIBAL in THF dropwise (6.2 mL, 6.2 mmol). The reaction was allowed to warm to 0° C. and stirred for 2 hrs. The reaction was quenched with 400 μL of H₂O and 400 μL of 15% NaOH and allowed to warm to room temperature. After 15 minutes, 1 mL of H₂O was added and was stirred for an additional 15 minutes. This slurry was then filtered over celite and washed with MeOH. The supernatant was concentrated under reduced pressure. The product was purified by flash chromatography (0-100% CH₂Cl₂/(30% MeOH/CH₂Cl₂)) over 18 minutes to afford a final product as a dark brown solid (20 mg, 8% yield). ¹H MeOD (400.13 mHz): 7.82 (s, 1H), 7.31 (d, J=8.5 Hz, 2H); 6.86 (d, J=8.5 Hz, 3H); 6.86 (d, J=8.5 Hz, 2H); 6.74 (dd, J₁=8.7 Hz, J₂=1.9 Hz, 1H); 6.58 (s, 1H); 4.57 (dt, J₁=47.2, J₂=5.9 Hz, 2H); 4.36 (t, J=6.5 Hz, 1H); 4.02 (t, J=6.2 Hz, 2H); 2.08 (m, 2H). ¹³C MeOD (100.6 mHz): 159.5, 146.3, 140.3, 135.7, 128.9, 115.6, 113.9, 81.7 (d, J=163.0 Hz), 64.8 (d, 6.0 Hz), 61.7, 49.6, 31.5 (d, 20.1 Hz); HRMS (ESI/Q-TOF) m/z: [M+H]+ Calcd for C18H21FN4O, 329.1772; Found 329.1770.

Synthesis of 1-(1H-benzo[d]imidazol-5-yl)-5-(4-(3-fluoropropoxy)phenyl) imidazolidin-2-one: Compound 8. To a stirring solution of 7 (101 mg, 0.31 mmol), triethylamine (125 mg, 1.24 mmol) in THF (20 mL) was added 1,l′-Carbonyldiimidazole (100 mg, 0.62 mmol) and stirred at 60° C. overnight. The reaction was cooled, diluted with H₂O and EtOAc. The product was extracted 3× with EtOAc. The organic layers were combined, washed with brine, dried over Na₂SO₄, filtered, and concentrated under reduced pressure. The product was purified by flash chromatography (0-100% CH₂Cl₂/(20% MeOH/CH₂Cl₂)) over 18 minutes to afford a final product as a white solid (38 mg, 35% yield). ¹H MeOD (600.13 mHz): 9.19 (s, 1H); 7.86 (s, 1H): 7.65 (m, 2H); 7.30 (d, J=8.7 Hz, 2H); 6.86 (d, J=8.8 Hz, 2H); 5.51 (m, 1H); 4.55 (dt, J₁=47.2 Hz, J₂=5.9 Hz, 2H); 4.01 (d, J=6.4 Hz, 2H); 3.97 (t, J=9.1 Hz, 1H); 3.33 (m, 1H); 2.07 (m, 2H). ¹³C MeOD (150.9 mHz): 161.9, 160.3, 140.8, 139.5, 133.3, 132.3, 129.0, 128.0, 122.1, 116.0, 115.0, 107.3, 162.9 (d, J=162.9 Hz), 64.8 (d, J=6.0 Hz), 61.8, 48.2, 31.4 (d, J=19.6 Hz).

HRMS (ESI/Q-TOF) m/z: [M+H]+ Calcd for C19H19FN4O2 355.1565; Found 355.1561.

Animals. A colony of 5XFAD mice obtained from Jackson Laboratories was maintained by crossing with WT C57BL/6J (McClure, R. et al. Aerosol Delivery of Curcumin Reduced Amyloid-beta Deposition and Improved Cognitive Performance in a Transgenic Model of Alzheimer's Disease. J Alzheimers Dis 2017, 55 (2), 797-811). The animals were genotyped by polymerase chain reaction (PCR) using DNA obtained from tail or ear tissue samples. After PCR amplification, DNA product was analyzed using a 1% agarose gel; amyloid precursor protein (APP) transgene=377 bp, and presenilin 1 (PSEN1) transgene=608 bp. 5XFAD mice were maintained as heterozygous. Animal experiments were conducted in accordance with the guidelines established by the Vanderbilt University's Institutional Animal Care and Use Committee (IACUC) and the Division of Animal Care and approved by Vanderbilt IACUC, protocol number M1700044.

Dynamic PET imaging. The dynamic acquisition was divided into twelve 5-second frames, four 60-second frames, five 120-second frames, three 5-min frames, and four 10-min scans. The data from all possible lines of response (LOR) were saved in the list mode raw data format. The raw data were then binned into 3D sinograms with a span of 3 and ring difference of 79. The images were reconstructed into transaxial slices (128×128×159) with voxel sizes of 0.03882×0.03882×0.0796 cm³ using the OSEM3D/MAP algorithm with 2 OSEM3D iteration followed by MAP 16 subsets, 18 iterations, beta of 1.47097, and MAP resolution of 1.5 mm. For anatomical co-registration, immediately following the PET scans, the mice received a CT scan in a NanoSPECT/CT (Mediso, Washington DC) at an X-ray beam intensity of 90 mAs and x-ray peak voltage of 45 kVp. The CT images were reconstructed into 170×170×300 voxels at a voxel size of 0.4×0.4×0.4 mm³. The PET/CT images were uploaded into Amide software (www.sourceforge.com) and co-registered to each other based on bed position and to an MRI template made in-house. Volumetric regions-of-interest (ROIs) were drawn around the cortex, hippocampus, striatum, thalamus, and cerebellum in addition to the whole brain in the template and superimposed onto the PET images. The PET images were normalized to the injected dose and animal weight, and the time-activity curves (TACs) of the mean activity within the ROIs were estimated for the entire duration of the scans in SUV (standard uptake values).

Glutaminyl Cyclase Inhibition Assays. The following is a modified assay based on the previously-reported work (Schilling, S. et al. Continuous spectrometric assays for glutaminyl cyclase activity. Anal Biochem 2002, 303 (1), 49-56) with some modifications. Human recombinant glutaminyl peptide cyclotransferase (QC) and human recombinant pyroglutamyl peptidase I (PGPEP1) were obtained from ProSpec-Tany TechnoGene Ltd. (Rehovot, Israel). PQ912 was purchased from Aobious Inc. (Gloucester, MA). PB-0822 was developed in-house as described elsewhere in this paper.

Assays were performed in 96-well plates at 37° C. in a buffer solution consisting of pH 6.0 HEPES with 1 mM dithiothreitol (DTT) and 20% (v/v) glycerol. H-Gln-AMC hydrobromide salt (Bachem Americas Inc, Torrance, CA) was used as the substrate. Each assay replicate contained 0.125 μg QC and 1.25 μg PGPEP1. After a 15-minute pre-incubation period for all reagents at 37° C. reactions were initiated by the addition of QC to a solution containing PGPEP1, 80 μM H-Gln-AMC, and varying concentrations (0-1000 nM) of PQ912 or either isomer of PB0822. Immediately after addition of QC, fluorescence emission at 460 nm (380 nmₑₓ) was measured every minute for 2 hours using a microplate reader (Biotek Industries, Agilent Technologies, Winooski, VT, USA). Data were exported to Excel, and fluorescence values were converted to product formation rate by using the equation generated from a standard curve made with 7-Amino-4-Methylcoumarin (Sigma Aldrich Inc, St. Louis, MO) under assay conditions. IC₅₀ values were calculated using an online calculator from AAT Bioquest (www.aatbio.com/tools/ic50-calculator).

Cardiac perfusion procedure and tissue collection. All IHC brain data were collected on perfused mice. Basically, deeply anesthetized mice were laid on a stainless-steel tray half filled with crushed ice, and the thoracic cavity was opened with a scalpel after making 5-6 cm mid-line incision starting from the abdominal area. After careful separation of the liver from the diaphragm, the thoracic opening was held open with the assistance of a retractor. Perfusion was commenced as described in the past (McClure et al. J Alzheimers Dis 2017, 55 (2), 797-811; McClure, R. et al. Inhalable curcumin: offering the potential for translation to imaging and treatment of Alzheimer's disease. J Alzheimers Dis 2015, 44 (1), 283-295; McClure, R. A. et al. Pham, W. Identification of promethazine as an amyloid-binding molecule using a fluorescence high-throughput assay and MALDI imaging mass spectrometry. NeuroImage: Clinical 2013, 2, 620-629) by slowly injecting the left ventricle with ice-cold PBS (1×) buffer pH 7.4 (30 mL) toward the ascending aorta using a 25-G syringe while the right atrium was quickly snipped off using a curved-point squeeze-snip scissors to facilitate drainage of the systemic venous return. After perfusion with PBS, the process was repeated with 4% paraformaldehyde (PFA, pH 7.4, 30 mL). When completed, brain and other tissues were harvested for preservation as described before (Barton, S. M. et al. Inhalable Thioflavin S for the Detection of Amyloid Beta Deposits in the Retina. Molecules 2021, 26 (4); Fuchter, M. et al. Clean and efficient synthesis of O-silylcarbamates and ureas in supercritical carbon dioxide. Chem Commun (Camb) 2008, (18), 2152-2154).

Immunohistochemistry. Brains collected from paraformaldehyde perfused mice were embedded in OCT and cut into coronal sections (8-10 μm) using a Tissue-Tek cryostat and mounted onto charged glass slides. Prior to staining, slides were washed with PBS (10 min); then, they were treated with blocking buffer (5% normal goat serum, 0.2% Triton X-100, 0.5% bovine albumin in PBS) for 1 h at room temperature. The treated sections were then incubated overnight at 4° C. with primary rabbit anti-pyroglutamate antibody (1:500 dilution, Novus Biologicals, Littleton, CO, USA, catalog number: NBP1-44048). Slides were washed with PBS (3×) for 10 min each, and the sections were subsequently incubated with secondary antibody goat anti-rabbit Alexa Fluor 647 (1:500 dilution, Thermo Fisher Scientific, Carlsbad, CA, USA, catalog number: A-21245) for 30 min at room temperature. The sections were then washed with PBS twice for 10 min and once for 30 min, and coverslipped with an antifade mounting medium (Vector Laboratories, Burlingame, CA, catalog number: H-1200-10) before observation under a fluorescence microscope.

Quantitative data analysis. Quantification of PET imaging and IHC signal were performed using imageJ software. Manual regions-of-interest (ROIs) were drawn and thresholded using identical parameters across samples before counting pixel intensity. Then, the data were imported to GraphPad Prism version 10 for Mac (Graphpad Software, San Diego, CA, USA) for statistical analysis. Significant differences between two independent groups were determined and compared using a paired parametric t-test. Significance is reported when the probability value <0.05.

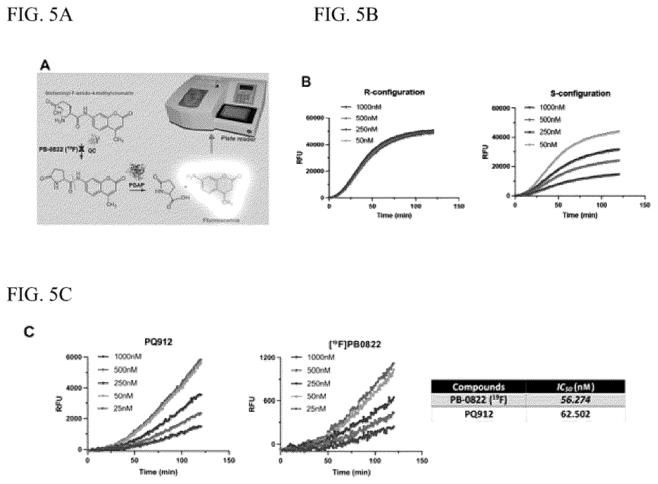

Synthesis of the precursor for [¹⁸F]fluoride labeling and the [¹⁹F] standard compound. In this optimized and reproducible scheme of synthesis (FIG. 3A), the reaction started with making the tosylate compound 1. A Strecker reaction was utilized in the next step between the aldehyde and the aminated benzimidazole in the presence of trimethysilyl cyanide (TMSCN) to afford the cyanomethylated amine 2 (FIGS. S3-4, SI). An overnight hydrogenation reaction enabled the reduction of nitrile into an amine 3 catalyzed by Pd/C at 120 psi. Finally, successful ring closure was achieved by treating aminated 3 with 1,1′-carbonyldiimidazole (CDI) to provide the desired precursor 4 with modest yield. The enantiomers of the precursor were separated using a chiral column (FIG. 3B), upon conversion to the [¹⁹F] versions, each enantiomer was subjected to the bioassay described in FIG. 5; only the S-configuration was recognized by the QC, henceforth only the S-configuration isomer is discussed hereafter.

The chemical design of this PET precursor is unique for two reasons. First, we decided to leave the amine on benzimidazole unprotected. During the course of this work, we found Boc-protected benzamidine is very labile; it can easily be removed with a trace of TFA present in the HPLC buffer. The active species emanated during Boc deprotection contributes to further destabilizing the compound. Second, the tosylate group was incorporated in the early synthetic steps. Initially, we were uncertain whether it will survive in the Strecker synthesis or during the hydrogenation. Particularly, deoxygenation of aryl tosylates under a palladium catalyst has been reported in the past. To our delight, the tosylate group survived both reactions. Aside from being the ideal leaving group for [¹⁸F]F⁻ labeling, incorporating of tosylates right from the beginning of the scheme has two goals, one is to serve as a protecting group for the hydroxyl moiety; second, tosylates have the propensity for forming crystals even with a few milligrams, offering an impeccable opportunity for characterization of the chiral products.

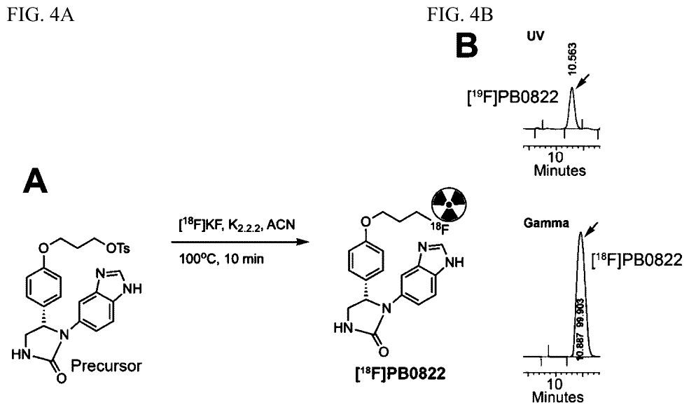

In a conventional approach to test the robustness of fluorine labeling with tosylate as a leaving group, particularly in the presence of free amines, precursor 4 was treated with tetra-n-butylammonium fluoride (TBAF) at room temperature to provide the product 8 with reasonable yield (detailed synthesis is in SI). However, large scale synthesis of [¹⁹F]PB0822 for use as a standard analog was achieved using identical chemical steps when obtaining the tosylate precursor 4, except 1-fluoro-3-iodopropane was used in the first step of the reaction instead of 1,3 bis-tosylate propane (FIG. 3A).

[¹⁸F]PB0822 radiotracer synthesis. The tosylate precursor was labeled with [¹⁸F]fluoride with an optimized condition, using [¹⁸F]KF and K_{2.2.2} in acetonitrile at 100° C. for 10 min with the unprotected benzimidazole (FIG. 4A). The radiochemical purity and the identity of the [¹⁸F]PB0822 PET radioligand were characterized by an analytical HPLC system, equipped with a UV absorption detector (λ=254 nm) and a radioisotope detector (Bioscan Flow-Count). The HPLC setup comprises a Phenomenex Luna 5 μm C18(2) (00G-4252-E0, 100 Å. 250×4.6 mm) column with a typical mobile phase of acetonitrile and ammonium formate (30%: 70% of 0.1 M, pH=6.5) at a flow rate of 1 mL/min. The identity of the [18F]PB0822 was confirmed by comparing the retention time with co-injected and standard compound [¹⁹F]PB0822 (RT=10.56 min) along with the gamma peak (RT=10.88 min) (FIG. 4B). The [¹⁸F]PB0822 was obtained with a 1.2% yield (non-decay corrected) at EOS with 99.9% radiochemical purity and with a molar activity of the radioligand at 965 Ci·mmol⁻¹.

The [¹⁹F]PB0822 standard compound retains comparable IC₅₀ value as of PQ912. The present inventors used a reported fluorescence assay (Schilling, S. et al. Continuous spectrometric assays for glutaminyl cyclase activity. Anal Biochem 2002, 303 (1), 49-56) with some modifications to assess the specificity among the isolated enantiomers of [¹⁹F]PB0822 for QC. The overall idea about this assay is depicted in FIG. 5A. As the amino group of coumarin dye (AMC) is incorporated into glutamic acid, the perturbation of the electronic propagation of coumarin results in quenching of the fluorescence signal. In the presence of QC, pyroglutamate-AMC is formed through cyclization of the N-terminal glutamate and the carboxylic side chain. Then, pyroglutamyl peptidase 1 (PGPEP1), an enzyme specific for pyroglutamyl, cleaves the substrate at the amide bond and releases the aminated coumarin, which restores fluorescence. In the presence of a specific QC inhibitor, QC activity will be hindered, resulting in reduced fluorescence output. The data showed that only [¹⁹F]PB0822 with S-configuration can attenuate the fluorescence in the assay, suggesting its targeted specificity for the QC enzyme (FIG. 5B). Furthermore, the probe inhibited QC activity at low-end nanomolar concentrations and in a dose-dependent fashion. In contrast, the assay confirmed that QC did not recognize the R-conformation, despite of testing under identical reaction conditions and concentrations as described for the S-counterpart.

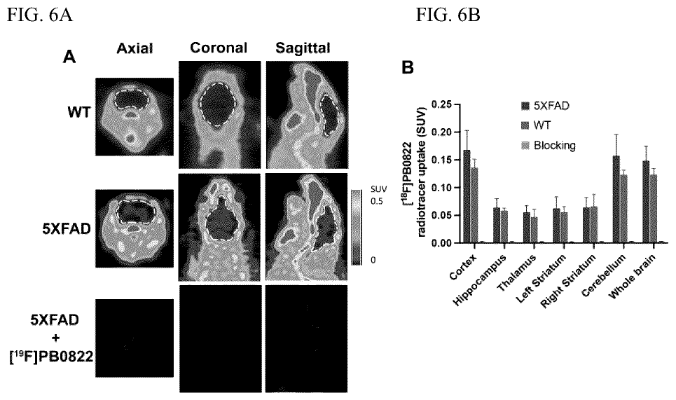

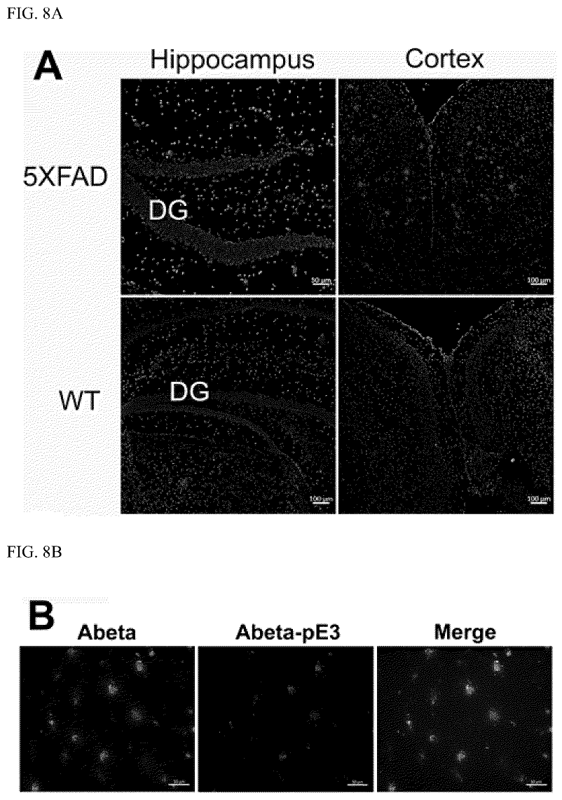

Fitting the tested concentrations of PQ912 and those of [¹⁹F]PB0822 in the regression model resulted in comparable IC₅₀ values for both compounds (FIG. 5C). The data suggested that adding a fluorine atom in PQ912 does not alter QC recognition, binding specificity, and potency.

[¹⁸F]PB0822 PET radioligand detects QC in the brains of 5XFAD mice. With the availability of a novel PET radioligand, we demonstrated for the first time noninvasive PET imaging data for the visualization of QC activity in the brain. In this triple-blinded study, animals' IDs and types were not revealed to the team that performed tail vein injection and imaging of animals, as well as to the imaging analysis team. The WT (n=5) and 5XFAD (n=11) mice received equivalent intravenous injection doses (400 μCi/0.1-0.2 mL) via the tail veins and were imaged immediately for a 75-minute dynamic scan or imaged 30 minutes after treatment for a 20-minute scan. In the 30 minutes post-injection cohorts, a higher PET signal was detected in 5XFAD brains as compared to WT brains, suggesting increased QC activity in an AD mouse model (FIG. 6A). Quantitative analysis of the SUV data showed that PET signal in 5XFAD brains was statistically higher than that of WT counterparts, in most of the brain regions (p=0.02) (FIG. 6B). To demonstrate the specificity of the probe, selected 5XFAD mice (n=3) were injected with “cold” compound of [¹⁹F]PB0822 (22.5 mM) 5 minutes prior to injection of the probe. After 30 minutes of uptake, animals were scanned, and the imaging data showed that the excess amount of “cold” compound competed with the probe, leading to a near abolishment of the PET signal in the brain (p=0.002) (FIGS. 6A,B). Taken altogether, the data suggest [¹⁸F]PB0822 reported the differential QC activity in the brains of normal versus pathological brains. This distinguishable signal intensity difference would likely provide even better signals in larger brain structures, such as those of non-human primates or humans. The in-vivo PET imaging in this blind study corroborates with human data reported earlier that QC is widely distributed in AD patients' cortex and hippocampus. This unique form of Abeta is a major constituent of Abeta deposits in sporadic and familial AD. The upregulated QC activity in AD patients correlated with the existence of large concentration of Abeta-pE3. Our PET imaging data using [¹⁸F]PB0822 show that 5XFAD mouse brains have higher levels of QC than those of WT counterparts. Particularly, we observed more QC activity in the cortex and cerebellum compared to those in the hippocampus. Furthermore, our immunohistochemistry data corroborates with PET imaging data, as it also showed higher levels of Abeta-pE3 in the cortex than hippocampus (FIG. 8). Our observation is consistent with reported data, indicating significantly enhanced QC activity in AD frontal cortex compared to neurological controls.

To test the time-dependent distribution to the brain, dynamic PET scans were obtained immediately after intravenous injection of 5XFAD mice (n=2) with [¹⁸F]PB0822 radioligand (400 μCi/0.1-0.2 mL). The data showed that the probe was distributed to the brain 5 minutes post-intravenous injection (FIG. 7). This early accumulation and retention of the probe is modest, yet abundant enough for detection. The time-activity curve (TAC) data showed that the uptake in the cortex and cerebellum is higher than other regions, suggesting QC activity might be more prominent in these brain subregions (not shown). While the whole-body PET imaging data indicated that imaging QC with this probe is unique because there was no indication of overwhelming background signal from peripheral tissues/organs (FIG. 7, lower panel). Aside from remarkable signal in the brain, there was also early detection of strong signal in the kidneys, 5-10 minutes post injection, suggesting some QC activity in the kidneys (FIG. 7, lower panel).

Copious presence of Abeta-pE3 found in the brains of 5XFAD mice. Coronal brain sections of approximately 8-10 μm thickness of WT (n=3) and 5XFAD (n=3) mice were stained with anti-Abeta-pE3 primary antibodies and visualized with a dye-labeled secondary antibody using a fluorescent microscope. The data indicated that there is no Abeta-pE3 in WT mouse brains (FIG. 8). In contrast, 5XFAD brains harbored significant levels of Abeta-pE3 in the brain. More Abeta-pE3 was detected in the cortex compared to the hippocampus. This regional distribution of Abeta-pE3 is the product of QC activity, which was observed in the in-vivo PET imaging data using [¹⁸F]PB0822.

Inhibition of QC activity is an ideal target for treating AD. A recent study showed that treating mouse models of AD with oral doses of a QC inhibitor resulted in reduced pyroglutamate Abeta burden, diminished plaque formation, and improved cognition¹⁸. Other work has shown that treating mice with anti-Abeta-pE3 monoclonal antibodies resulted in the attenuation of behavioral deficits and clearance of Abeta in preclinical mouse models³⁸. Taking all of these promising data into account, the present invention meets a long-felt need by developing imaging technology to help to assess these observations non-invasively. Furthermore, embodiments of the present invention will help to speed up the screening of QC inhibitors and evaluate the efficacy of these drugs in clinical trials. Since QC is involved in the early onset of AD, the present invention can potentially help to stratify AD patients admitted to clinical trials; thus, it could hold considerable benefits for future AD diagnosis, prognosis, management, and treatment.

In summary, compounds of the present invention are ideal PET radioligands for imaging QC activity in AD. Further, these probes can be translated to humans. Aside from specificity for QC, the probes of the present invention have an acceptable solubility profile, enabling formulation for in-vivo applications. Furthermore, the probes of the present invention can cross the blood-brain barrier and pinpoint QC in the brains of 5XFAD mice.

All publications, patents, and patent applications mentioned in this specification are herein incorporated by reference to the same extent as if each individual publication, patent, or patent application was specifically and individually indicated to be incorporated by reference.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound represented by Formula I, a hydrate, a solvate, a pharmaceutically acceptable salt, or combination thereof, wherein 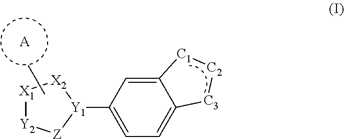
A is a 5- or 6-membered aryl ring, or a 5- or 6-membered heteroaryl ring;
a is 0 to 5;
X₁ and X₂ are each independently O, S, C(Rₓ)₂, —C(Rₓ)₂—C(Rₓ)₂—, —C(Rₓ)₂—O—, wherein at least one of X₁ and X₂ is not —C(Rₓ)₂—C(Rₓ)₂— or —C(Rₓ)₂—O—;
Y₁ is CH, CH(R_{y}), or N;
Y₂ is CH₂, CH(R_{y}), C(R_{y})₂, O, S, NH, or NR₁₀;
Z is C(R_{z})₂ or Z′(═X′)_{x′}, wherein x′ is 1 or 2;
Z′(═X′)_{x′} is C(═O), C(═CH(R_{x′})), C(═C(R_{x′})₂), C(═S), S(═O), or S(═O)₂;
B₁, B₂, and B₃ are each independently CH, C(R_{b}), or N;
C₁, C₂, and C₃ are each independently CH, C(R_{c}), or N;
each occurrence of Rₓ is independently hydrogen or a substituent;
each occurrence of Rₐ, R_{b}, R_{c}, R₁₀, R_{y}, and R_{z} is independently a substituent;
each occurrence of a substituent is:
hydroxyl, deuterated hydroxyl, thiol, deuterated thiol, cyano, halogen, isonitrile (—NC), tri C₁-C₁₂ alkylammonium, ketone, sulfonate, or nitro; or
a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), optionally substituted with a deuterium, hydroxyl, deuterated hydroxyl, thiol, deuterated thiol, cyano, halogen, isonitrile (—NC), tri C₁-C₁₂ alkylammonium, ketone, sulfonate, or nitro, or a combination thereof,
wherein any carbon-carbon single bond of the C₁-C₁₂ alkyl and the C₁-C₆ alkyl is optionally replaced by at least one carbon-carbon double or triple bond, and any methylene of the C₁-C₁₂ alkyl and the C₁-C₆ alkyl is optionally replaced by at least one O, S, NR₁₀, oxo (—C═O), imido (—C═NR₁₀), thioxo (—C═S), sulfoxo (S═O), sulfone (S(═O)₂), Se, Ge, or Si;
wherein (i) the compound is an imaging agent and comprises a detectable halogen group; or (ii) the compound is a synthetic precursor of an imaging agent comprising a detectable halogen group.

2. The compound of claim 1, wherein the compound is a synthetic precursor of an imaging agent and comprises a leaving group comprising cyano, halogen, isonitrile (—NC), tri C₁-C₁₂ alkylammonium, C₁-C₁₂ alkyl ketone, sulfonate, or nitro.

3. The compound of claim 2, wherein the leaving group is a sulfonate group.

4. The compound of claim 1, wherein the compound is an imaging agent comprises at least 95% radiochemical purity as determined by analytical HPLC, equipped with a UV absorption detector and a radioisotope detector.

5. The compound of claim 1 represented by one of Formulas IIA-IIB

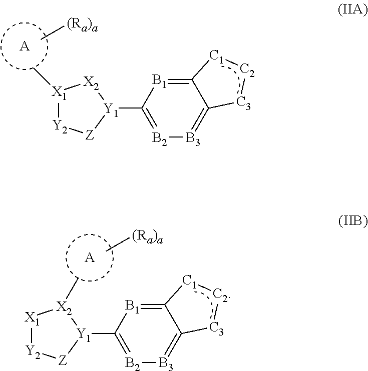

5. The compound of claim 1, wherein
Z is Z′(═X′)_{x′}; and
Y₁ is N; or
Y₂ is NH or NR₁₀.

6. The compound of claim 1, wherein Z is Z′(═X′)_{x′}, wherein Z′(═X′)_{x′} is C(═CH(R_{x′})), C(═C(R_{x′})₂), S(═O), or S(═O)₂.

7. The compound of claim 1 represented by one of Formulas IIIA-1, IIIA-2, IIIB-1, and IIIB-2: 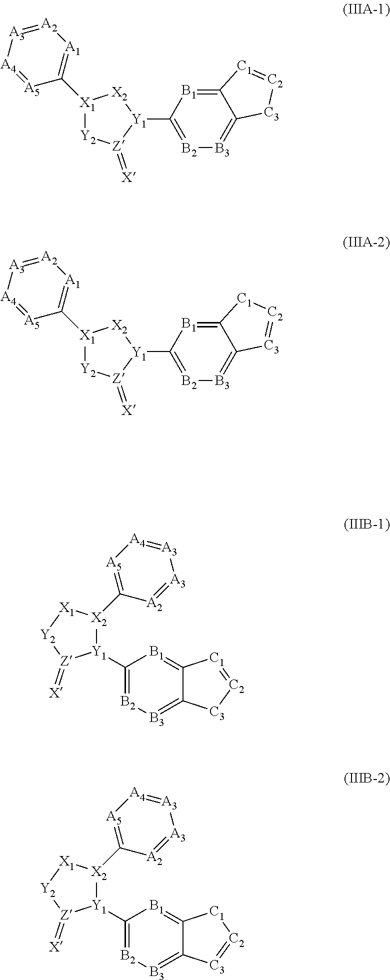
wherein A₁-A₅ are each independently CH, C(Rₐ), or N.

8. The compound of claim 1, wherein
i) the compound is a synthetic precursor and at least one of Rₐ, R_{b}, or R_{c} is present and comprises: a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), each substituted with a sulfonate group, nitro, trialkyl ammonium salt, Br, CI, I, ¹⁹F, CN, CF₃, or ketone (C(═O)alkyl), and each optionally substituted with a deuterium, wherein any methylene is optionally replaced by at least one O, S, NR₁₀, oxo (—C═O), imido (—C═NR₁₀), thioxo (—C═S), sulfoxo (S═O), sulfone (S(═O)₂), Se, Ge, or Si;
ii) the compound is an imaging agent comprising a detectable halogen group and at least one of Rₐ, R_{b}, or R_{c} is present and comprises one of the following groups comprising a detectable halogen group: a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), each optionally substituted with a deuterium, a no-radioactive halogen, or a combination thereof, wherein any methylene is optionally replaced by at least one O, S, NR₁₀, oxo (—C═O), imido (—C═NR₁₀), thioxo (—C═S), Se, Ge, or Si.

9. The compound of claim 1 represented by one of Formulas IVA, IVB, IVC, and IVD:

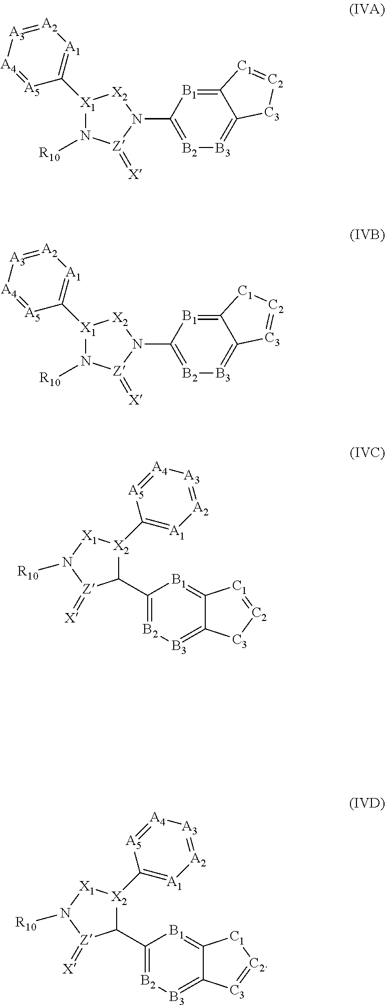

10. The compound of claim 9, wherein at least one of conditions i)-iii) is true:
i) at least one of A₁-A₅ comprises C(Rₐ), wherein Rₐ comprises formula -Lₐ-W;
ii) at least one of B₁-B₃ comprises C(R_{b}), wherein R_{b} comprises formula -Lₐ-W;
iii) at least one of C₁-C₃ C(R_{c}), wherein Re comprises formula -Lₐ-W;
wherein Lₐ is a single bond, Lₐ₁, or Lₐ₂;
Lₐ₁ is:
a single bond, a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), optionally substituted with a deuterium, a halogen, or a combination thereof, wherein any methylene is optionally replaced by at least one O, S, NR₁₀, oxo (—C═O), imido (—C═NR₁₀), thioxo (—C═S), sulfoxo (S═O), sulfone (S(═O)₂), Se, Ge, or Si;
Lₐ₂ is represented by formula U-L_{a2′}, wherein
U is O or S, and
L_{a2′} is a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), optionally substituted with a deuterium, a halogen, or a combination thereof;
and
W is a sulfonate, cyano, halogen, isonitrile (—NC), tri C₁-C₁₂ alkylammonium, C₁-C₁₂ alkyl ketone, or nitro; or a detectable halogen group.

11. The compound of claim 10, wherein Lₐ₁ and L_{a2′} each independently comprise a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₂-C₆ heterocycloalkyl, a C₂-C₆ heterocycloalkenyl, a C₁-C₆ alkyl(C₂-C₆ heterocycloalkyl), a C₁-C₆ alkyl(C₂-C₆ heterocycloalkenyl), a C₅-C₁₂ aryl, C₂-C₃₀ heteroaryl, a C₁-C₆ alkyl (C₅-C₁₂ aryl), or a C₁-C₆ alkyl (C₂-C₃₀ heteroaryl), optionally substituted with a deuterium, a halogen, or a combination thereof.

12. The compound of claim 11, wherein
Lₐ₁ and L_{a2′} each independently comprise a C₁-C₁₂ alkyl, a C₂-C₁₂ alkenyl, a C₂-C₁₂ alkynyl, a C₃-C₆ cycloalkyl, a C₁-C₆ alkyl(C₃-C₆ cycloalkyl), a C₁-C₆ alkyl(C₃-C₆ cycloalkenyl), a C₃-C₆ cycloalkenyl, a C₅-C₁₂ aryl, a C₁-C₆ alkyl (C₅-C₁₂ aryl), each optionally substituted with a deuterium, a halogen, or a combination thereof.

13. A kit comprising components A and B, wherein component A is a compound of claim 1, comprising a synthetic precursor of an imaging agent, and component B is a radioactive isotope source.

14. A pharmaceutical composition comprising a compound of claim 1 and a pharmaceutically acceptable excipient.

15. A method form preparing a compound of claim 1 comprising a detectable group, wherein the method comprises reacting a compound of claim 1 comprising a leaving group under conditions effective to provide the compound comprising a detectable group, preferably ¹⁸F.

16. An imaging method for detecting amyloid β in a subject, comprising administering an effective amount of a compound of claim 1 comprising a radioactive isotope to the subject.

17. The method of claim 16, wherein the imaging method is PET and the radioactive isotope is ¹⁸F.

18. A method for monitoring the severity of a disease or disorder associated with the presence of amyloid peptides, tau proteins of neurofibrillary tangles, or a combination thereof in a subject, wherein the method comprises administering to the subject an effective amount of the compound of claim 1 comprising a detectable group.

19. The method of claim 18 wherein the disease or disorder is a neurological disease or a kidney disease and the detectable isotope is ¹⁸F.

20. The method of claim 19, wherein the disease or disorder is Alzheimer's Disease.
